(19) United States
(12) Patent Application Publication
(10) Pub. No.: US 2022/0387796 A1
(43) Pub. Date: Dec. 8, 2022

(54) ELECTRICAL STIMULATION METHOD FOR MEDICAL TREATMENT

(71) Applicants: Richard MALTER, Frankston (AU); Sujit NAGARAJAN, Waterways (AU)

(72) Inventors: Richard MALTER, Frankston (AU); Sujit NAGARAJAN, Waterways (AU)

(21) Appl. No.: 17/817,956

(22) Filed: Aug. 5, 2022

Related U.S. Application Data
(63) Continuation-in-part of application No. PCT/AU21/00011, filed on Feb. 8, 2021.

(51) Int. Cl. A61N 1/36; A61N 1/04

(52) U.S. Cl. CPC A61N 1/36031; A61N 1/0492

(57) ABSTRACT

A multi-type combination electrical stimulation method is for medical treatment of surface and internal infected tissues, surface wounds and ulcers, trauma injuries, abnormal plasia and fibrotic changes, and pain conditions. The multi-type electrotherapy system for non-invasive treatment of both surface and deep body bacterial and viral infections and wound healing includes a machine learning function optimization task algorithm, and a stimulation device electronically with the ability to generate carrier base waveforms with amplitude modulation of these waveforms by secondary frequencies. The ranges of such frequencies are base frequencies in the range of 1-20,000 Hz and modulating frequencies in the range of 1-200 Hz. The waveforms are generated by either direct digital synthesis (DDS) or digital to analogue (DAC) converter electronics.

### CROSS-REFERENCE TO RELATED APPLICATIONS

The present application claims priority under 35 U.S.C. Section 120 from U.S. Patent Application Serial No. PCT/AU21/00011, filed on 8 Feb. 2021, entitled “FUNCTION OPTIMIZATION ALGORITHM AND MULTI-TYPE ELECTROTHERAPY COMBINATION TREATMENT”. See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a dynamic electrical stimulation treatment method and device for multi-type electrotherapy combinations that includes stimulation of intracellular second messengers. The present specification also discloses an iontophoresis and low intensity direct current (LIDC) device and method of treatment.

### 2. Description of Related Art Including Information Disclosed Under 37 CFR 1.97 and 37 CFR 1.98

Electrotherapy, also termed electromedicine and electrical stimulation (ES), and in neurostimulation applications, electroceuticals, has many types, modalities and variations thereof including but not exhaustively: TENS, MENS, NMES, FES, pulsed electromagnetic field (PEMF) stimulation, direct current (DC) stimulation including low and ultra-low intensity stimulation, AC and other waveform generating stimulation with or without secondary amplitude or frequency modulation, electroacupuncture, microcurrent, interferential (IF), transcranial stimulation devices, targeted neural circuit stimulation, silver-nylon cloth electrode iontophoresis stimulators (SIS) and low intensity direct current (DC) and DC electric field (EF) stimulation as disclosed in patent AU 2016202751, targeted ES for promoting bone fracture healing and nerve tissue regeneration, and liquid medication iontophoresis also known as transcutaneous drug delivery that is a hybrid of electrotherapy and drug treatment, all with specific and intended target therapeutic effects.

Due to the enormous complexities and rapid spatio-temporal dynamics of tissue, cellular, and molecular (including genetic) microenvironments, and the effectively unlimited combinations of pathological abnormalities that can occur within one or many of these microenvironments, it is extremely difficult to select an electrotherapy modality at any point in time, and that is to be continued for any extended period of time (seconds, minutes, hours, days), with reliable and reproducible prediction of significant therapeutic effect and of no detrimental effect on any localized target tissue. Additionally, it is impossible to be aware of the large and fast growing body of published medical ES literature with tens of thousands of articles reporting complex effects and data in a very large field of therapeutic and research applications and know how to apply this large body of information to specific medical case instances.

Yet a further obstacle to selecting single and combination ES modalities, is that the global level of knowledge and understanding of the mechanisms of action of the various ES modalities is still in its infancy with far more remaining unknown than established information within the ES medical-scientific literature.

Given the complexities and dynamics of both normal physiological processes and their pathological abnormalities all of which are active in very short timeframes, combinations of electrotherapy modalities are sometimes applied sequentially or simultaneously in order to supplement, augment, replace, provide energy to, up-regulate or otherwise variously stimulate multiple rather than single therapeutic targets and processes within the pathological target tissue microenvironments. Some specialized electromedical devices are capable of multiple ES modality combinations, with one example disclosed in patent AU 2016202751 where surface wound healing electrical stimulation is provided by a combination of low intensity DC (LIDC), EF stimulation, and by cell phenotype modification stimulation. However, selecting a therapeutically effective, and non detrimental, combination of ES modalities at any point in time during the acute to chronic stages of any pathology, normalization and healing—where a chronic or non healing surface wound or ulcer is one such example—remains practicably unworkable as being predictively from extremely difficult to impossible.

Iontophoresis is a process in which ions flow diffusively in a medium driven by an applied electrical field. The present disclosure also shows an electronic Device for the infusion of silver ions in medical iontophoresis.

Iontophoresis involves the interaction between ionized molecules of a source and an external electric field, resulting in the migration of charged cations or molecules. The migration is achieved by placing two electrodes on the patient's skin which are connected to a low intensity direct current (LIDC) power supply. One of the electrodes is a source or ‘Positive’ electrode. The other electrode is a ‘Return’ electrode. The Positive and Return Electrodes are effectively a positively charged anode and a negatively charged cathode, respectively. The electric potential and EF generated between the two electrodes causes the charged cations or molecules to migrate from the Positive Electrode directly into the tissues of the patient aligned between and directly towards the Return Electrode without the necessity of hypodermic injection and its adverse effects, such as pain and risk of infection, nor the shortcoming of infusions that depend on blood supply to reach target (infected) tissues—that may be remote from the site of the injection and/or be surrounded by micro-circulatory disturbances reducing or inhibiting drug or substance uptake.

The present invention seeks to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### BRIEF SUMMARY OF THE INVENTION

The invention in one aspect provides a multi-type electrotherapy system for non-invasive treatment of one or more of: surface and internal infected tissues, gross tissue injuries, abnormalities and morphological changes, surface wounds and ulcers, and pain conditions, the system comprising:
a machine learning algorithm, and
an electrical stimulation (ES) device electronically designed and software programmed with the learning algorithm and to include the ability to generate carrier base waveforms with amplitude modulation of the carrier base waveforms by secondary frequencies.

In one embodiment, the machine learning algorithm is a dynamic function optimization task algorithm.

In another embodiment, input data for the learning algorithm includes therapeutically desired vector quantity changes of a target bioelectric or biochemical parameter (Pbio) that is monitored and measured repeatedly electronically or chemically every few seconds or minutes.

In another embodiment, assessment of Pbio change is performed with continuously updated regression analysis or by other statistical and analytical means.

In another embodiment, the ranges of generated carrier base waveform frequencies by the ES device are base frequencies in the range of 1-20,000 Hz and the secondary frequencies are modulating signal frequencies in the range of 1-200 Hz.

In another embodiment, the waveforms are generated by either direct digital synthesis (DDS) or digital to analogue (DAC) converter electronics.

In another embodiment, the amplitude modulated waveforms generated by the electronic circuits of the stimulation device are utilized to stimulate and regulate specific intracellular second messengers.

In another embodiment, the second messengers include cyclic AMP: adenosine 3′, 5′-monophosphate (cAMP), and cyclic GMP: guanosine 3′,5′-cyclic monophosphate (cGMP).

In another embodiment, the stimulation device can operate as a constant voltage source outputting the DDS or DAC generated amplitude modulated waveforms that results in clinically effective ES.

In another embodiment, ES is performed through dense tissues and bone at very low Output Voltages.

In another embodiment, the Output Voltages are at the lower end of the millivolt range.

In another embodiment, for intracellular second messenger pathway stimulation, the software of the stimulation device generates a repeating, timed stimulation cycle that includes an up-regulating component that increases the production of a specific second messenger.

In another embodiment, the up-regulating component step is followed by a first rest period of variable programmable duration, then followed by a component that stimulates the activation and utilization of the specific second messenger

In another embodiment, the activation component step is followed by a second rest period also of programmable variable duration, after which the complete second messenger stimulation cycle then repeats from the beginning.

In another preferred embodiment, the base and modulating signal frequencies are: 4000 Hz amplitude modulated by 10 Hz for up-regulating production, followed by 4000 Hz amplitude modulated by 20 Hz for increasing activation and utilization of cAMP, and, 4000 Hz amplitude modulated by 25 Hz for up-regulating production, followed by 4000 Hz amplitude modulated by 20 Hz for increasing activation and utilization of cGMP.

In another embodiment, the first rest period following the production component and the second rest period following the activation component of the stimulation cycle are typically of 1-3 minutes duration in order to increase the overall biostimulation effect by allowing the electrochemical intracellular second messenger processes to physiologically synchronize with the stimulation cycles, and which have been found clinically to be maximally effective.

In another embodiment of the present invention the ES device is applied for electroacupuncture treatments based on either traditional theories or on modern neuroanatomy and neurophysiology. In this aspect of the present invention, the carrier base frequencies at the upper end of their 1-20,000 Hz range are utilized to match the frequency-dependent impedance profile of the internal body tissues through which the resultant current is induced lying between either the pair of cutaneous electrode pads or the inserted acupuncture or electromyograph needles that are connected to the output terminal of the ES device. And simultaneously, the secondary modulating signal frequencies at the lower end of their 1-200 Hz range are utilized to match the conduction velocity rates or their harmonic frequencies of the various nerve fiber types (A-delta, C, A-beta) for stimulating those nerve fibers, or for simulating their activation and signal transmission. Both of these necessary conditions for effective ES are absent or under considered in the prior art.

In another embodiment of the present invention applied to electroacupuncture, the lower end of the 1-20,000 Hz range base frequencies can be utilized to match neuronal refractory periods for attenuating or blocking pain nerve signals.

In another embodiment, all possible multi-type ES combinations are derived from a switch set table of base data comprising top level binary switch states of stimulation on and off, and non binary second level switch states consisting of the full ranges of each of the different ES types.

In another embodiment, the function optimization task algorithm tests, scores and selects ES switch sets based on their relative effects or not on Pbio.

In another embodiment, the learning algorithm continuously stores, updates and accesses the tested switch set scoring history to improve its switch set selection for performance of task optimization.

In another embodiment, the ferroelectric RAM memory of multiple ES devices are a continuously accumulating, shared knowledge-base, uploaded wirelessly and automatically from each ES device to a central or distributed electronic database that each new and older device accesses and utilizes in its learning algorithm.

In another aspect, a combined instance-based and regression algorithm and software code embodiment that is based on a partly true-random, dynamic function optimization task model. According to the definition that machine learning is the artificial intelligence (AI) field of the construction of algorithms and computer programs that automatically improve with their experience of performing tasks, the algorithm is a machine learning type.

Another aspect of the present invention is the application of a machine learning algorithm and executing software program to multi-type electrotherapy modality and stimulation combinations output by a single or multiple treatment devices.

In another aspect of the present invention, the environmental input data for the learning algorithm are the therapeutically desired vector quantity changes of a target bioelectric or biochemical parameter (Pbio) that is monitored and measured repeatedly electronically or otherwise every few seconds or minutes. Assessment of Pbio dynamics is performed with continuously updated regression analysis or by other statistical and analytical means.

In one aspect of the present invention, Pbio is the complex electrical impedance (Z), capacitive reactance (Xc), inductive reactance (XL) or phase angle (F) profiles across a frequency range sweep of any target tissue(s) that is anatomically localizable for cross sectional planar electronic measurement, invasively or non-invasively.

In another aspect of the present invention, when the function optimization algorithm is specifically applied to wound healing, Pbio is the complex electrical impedance (Z), capacitive reactance (Xc), inductive reactance (XL) or phase angle (F) measured through or across the wound or ulcer.

In another aspect of the present invention, when the function optimization algorithm is specifically applied to conditions of pain, especially those being mostly neuropathic type pain conditions, Pbio is the complex electrical impedance (Z), capacitive reactance (Xc), inductive reactance (XL) or phase angle (F) sweep profiles of the involved pathological nerve fibers and their individual types including C, A-delta, A-beta, and their corresponding diameters and myelinations.

In another aspect of the present invention, when the function optimization algorithm is specifically applied to fibrotic or cirrhotic tissues, Pbio is the complex electrical impedance (Z), capacitive reactance (Xc), inductive reactance (XL) or phase angle (F) sweep profiles of the targeted gross pathological tissues.

In another aspect of the present invention, when the function optimization algorithm is specifically applied to the treatment of neoplasms, Pbio is the electrical resistance or complex electrical impedance (Z), capacitive reactance (Xc), inductive reactance (XL) or phase angle (F) sweep profiles of the targeted neoplastic tissue mass.

In another aspect of the present invention, Pbio is the through-wound electrical resistance and endogenous wound generated electric field of a surface wound or ulcer used as a measure of wound healing and closure.

In another aspect of the present invention, multiple Pbios can be simultaneously assessed by duplicating and/or adapting sections of the function optimization task algorithm.

In another aspect of the present invention, the electronic stimulation device and its software program are capable of multiple ES modalities and stimulations, five of which are termed as illustrative examples and for convenience, ES-A, ES-B, ES-C, ES-D and ES-F, although more numerous ES modality/capabilities including any of those mentioned in the Background can be output by the device and software program.

In another aspect of the present invention, the function optimization task model can simultaneously optimize the functions of multiple ES modalities and stimulations.

Another aspect of the present invention is that each ES modality that the stimulation device and software are capable of has two top level binary switch states of being either switched on and output by the stimulation device or switched off and not output by the stimulation device, which further results in the feature that when the different ES modalities are output sequentially, this series combination digital stimulation method is also capable of producing analogue combination ES patterns if one or many of the ES modalities continues to be switched off. For example, if ES-A, ES-B, ES-C and ES-D are all switched off in one stimulation combination that is repeatedly output by the stimulation device, then modality ES-F will be output as a continuous analogue type ES.

Another aspect of the present invention is that to achieve the function optimization task the algorithm randomly at first generates and controls the electronic circuitry of the stimulation device to output one after another every possible sequential combination of ES-A, ES-B, ES-C, ES-D & ES-F, where one such example would be, ES-A_ON, ES-B_ON, ES-C_OFF, ES-D_OFF, ES-F_OFF, and where each such sequential combination thereby forms and is hereafter referred to as a switch set; and that each switch set is only output for a relatively short timeframe that is selected in relation to the biologically possible rate of variability and characteristics of Pbio, and the generation and output of all possible switch sets generated by the algorithm from all available switch state combinations is also completed within a similarly short biological timeframe also directly relative to the possible biological dynamics of Pbio.

In another aspect of the present invention the ES modalities that the stimulation device is capable of can have additional, second-level and lower level non binary switch states that allows the function optimization algorithm to generate more ES combination switch sets than the switch sets that comprise only the top level binary switch states of the ES modalities. For example, ES-A can have a range of electrical output parameter values of current, voltage or frequency depending on its ES type, where in the example of ES-A being constant Output Current with three possible discrete intensities, then ES-A can have the additional second-level switch states of, ES-A-1, ES-A-2 and ES-A-3; whereas in reality more numerous second-level switch states for ES-A can correspond to a preprogrammed or algorithm generated, gradated intensity step size through the entire range of minimum to maximum Output Currents that the stimulation device is capable of.

In another aspect of the present invention, the second-level switch states of constant Output Currents that correspond to gradated intensity steps through the constant Output Current range capability of the stimulation device from low intensity direct current (LIDC) up to milliampere intensity direct current (MIDC), add the capability to the function optimization algorithm to perform an automated search through the entire constant Output Current range of the stimulation device and find a second-level switch state that corresponds to a specific intensity LIDC or MIDC that has a therapeutic effect on the target localized tissue measured in terms of the positive therapeutic effect of that switch state on Pbio according to the assessment methodology of the function optimization task described above. The medical utility of this aspect of the present invention is that LIDC and MIDC can variously be applied therapeutically for the purpose of attenuating and inactivating the infection process in localized target tissue microenvironments, as for example disclosed with LIDC ES outputs in patent AU 2016202751 where a number of microorganism taxonomic-specific bell curve characterized effect-relationships were provided for viral and bacterial species. Though given the extremely large diversity within and among the different species and families of microorganisms including bacteria, protozoa, viruses, fungi and yeasts and that mutations of these microorganisms are possible and common, and because of the virtually unlimited complexities and dynamisms of tissue, cellular, and molecular microenvironments as noted above, preprogrammed Output Currents that previously interacted effectively with these target tissue microenvironments to attenuate and stop the infection process involving specific microorganisms might later on in similar specific medical case instances, be ineffective. Whereas, in contrast, the solution provided by the automated search performed by the function optimization algorithm of the present invention using non binary second-level switch states of constant Output Currents can find non predicted, unknown, variable and unique-to-instance therapeutically effective Output Current intensities for theoretically any type of tissue having a microenvironmental physiological abnormality including but not limited to, bioelectric state and pathological condition involving an infection process

In another aspect of the present invention the algorithm generated ES switch sets comprising only top level switch states and those comprising mixed level switch states are assessed dynamically in realtime for therapeutic success based on their performance effect on the continuously incoming Pbio data. The rate and limits of effect on Pbio by a switch set under test (SSUT) are computed by the algorithm as variables of the function optimization task model in order to determined if the SSUT is therapeutically successful or not. Score metadata are computed and assigned to a successful switch set that are then recorded and logged, and the switch set is then assigned as the active switch set (ASS) to be continuously output for therapeutic stimulation. While a switch set remains the ASS the algorithm continuously updates the score metadata of that ASS with experience from the stream of incoming Pbio data and its effect thereon.

In a preferred embodiment of the present invention, ASS score metadata are absolute, percentage and rate of change calculations of Pbio over pre-defined or variable time periods selected by the algorithm in relation to the involved physiological and pathological processes involving Pbio, the logged dynamics of Pbio prior to assigning the current ASS, duration of maintained therapeutic effect of the ASS on Pbio, and positional data of Pbio within its known or computed biological range of values in relation to the start and end points, and duration of the current ASS, and the specific pathology being treated.

Another aspect of the present invention is that when a successful switch set that has been assigned as the current ASS is no longer therapeutically effective at any point in time as determined by the assessment methodology already described, the function optimization algorithm first compares and matches the logged score metadata of previous ASSs to the recent dynamics and positional value of Pbio in order to predict their repeat successes of therapeutic effect on Pbio. When a previously assigned ASS is selected in this way and then repeats its success in terms of present therapeutic effect on Pbio then it is again assigned as the current ASS, and a new record of its score metadata is made; such that a single switch set can have multiple ASS assignments and corresponding records each having different score metadata. If all previous ASSs selected in this way are retested but are unsuccessful in terms of their present therapeutic effect on Pbio then the function optimization algorithm again randomly generates all possible ES switch set combinations that have not previously been assigned as ASSs and outputs and assesses each one in turn via the same methodology already described.

A resultant aspect of the present invention is that the overall function optimization task algorithm repeats until a new ASS is found.

Another key aspect of the present invention is that the function optimization algorithm that controls the combination ES switch sets output by the stimulation device, continuously learns and improves its assessment and predictive ability of what switch sets will be more or less effective at any point in time within the biological range and given the present and previous dynamics of the target Pbio from continuously incoming Pbio data and computations thereon of the current medical case it is applied to.

A further aspect of the present invention is that ASSs recorded during the application of the stimulation device and treatment method to one medical case instance, are permanently stored in the device's ferroelectric RAM (FRAM) memory as best shown in step MLO of FIG. 5 MULTI TYPE ES WOUND HEALING FLOWCHART. When the device is then applied in the future to the same or similar medical condition in another medical case instance, it first utilizes its knowledge of previous ASSs before needing to test other switch sets for therapeutic success on Pbio in the new medical case instance. As the number of the device's applications to more of the same or similar medical conditions increases, so does the device's knowledge, experience and speed in finding successful switch sets (ASSs). Thus the stimulation device learns and improves its therapeutic skill level with its own accumulating experience, which is permanently stored in its FRAM memory and continuously updated during each new medical case instance application, for ready access to any future medical case instance.

In another embodiment of the present invention, the FRAM memory of multiple devices are a continuously accumulating, shared knowledge-base, uploaded wirelessly and automatically from each device to a central or distributed electronic database that each new and older device accesses. Data is uploaded and retrieved by each device in realtime In this data storage and access embodiment of the present invention, the electronic database can have multiple areas of specialization for different medical conditions, such as but not limited to surface and deep body wounds and ulcers of all kinds, lacerations, hyper-, meta- and neo-plastic lesions, fibrotic tissue, and infections of various kinds.

In another aspect of the present invention, given the individual and heterogeneous nature of many types of medical problem even when similarly classified in medical nomenclature for conventional diagnosis and identification purposes—where the reality of the uniqueness of every surface wound or ulcer is an example—advance training data for the function optimization task model of the algorithm are not applicable nor possible, and so instead and preferably the software program acquires all of its training and learning data individually and uniquely while the stimulation device is applied to each individual medical case instance.

In a variant embodiment of the present invention when there is far greater homogeneity of the medical issue and also of the related characteristics and behaviour of the selected Pbio, then training data can be used to pre-train the function optimization algorithm.

Second Messenger Stimulation

In another aspect of the present invention, in order to further overcome the obstacles to predictable and reproducible therapeutic effect with ES, the stimulation device is electronically designed to include the ability to generate ‘carrier’ base waveforms with amplitude modulation of these waveforms by secondary (‘envelope’) frequencies also termed, signal frequencies, which are often the actual active element of the ES modality. Typical ranges are base frequencies from 1-20,000 Hz and modulating frequencies from 1-200 Hz, though these ranges are given for illustration only and are not limits to the present invention. The base frequencies are selected in order to use to advantage the complex impedance properties—particularly the bioelectrical capacitive reactance (Xc)—of the superficial tissues to the deeper localized target pathological tissues even if the superficial tissues contain thick bones, to enable signal frequency transmission, and to match the frequency-dependent impedance profile of the internal body tissues lying between the cutaneous electrode pads in which the resultant current is induced.

The internal body tissues impedance matching aspect of the present invention is illustrated by one example in supplementary FIG. 7 that shows the in vivo complex impedance (Z) sweeps between two SIS AgN electrodes together with simultaneous direct current (DC) resistance measurements made in parallel with the stimulation waveform generation circuit at a low millivolt Output Voltage. FIG. 7A shows the complete frequency range Z sweep and FIG. 7B shows the same Z sweep up to the approximately 5000 Hz range. Referencing Ohm's law for the general case of alternating current (AC) circuits, V=IZ, these Z sweep data show the need to use a base carrier frequency starting at approximately 4000 Hz in order to induce an internal current with a constant-current intensity characteristic with any given Output Voltage, in the millivolt range as graphed. At lower base frequencies, the Z value has a highly variable frequency dependence Additionally, the data show that it would be impossible to induce a low or very low internal body signal frequency with a constant-current parameter without the utilization of an impedance matched base (carrier) frequency. The accurate inducing of an internal body, low or very low frequency AC signal having a high precision constant-current parameter for achieving a highly specific targeted therapeutic effect is a key feature of this aspect of the present invention

In another aspect of the present invention the waveforms are generated by either direct digital synthesis (DDS) or digital to analogue (DAC) converter electronics. The advantages provided by DDS waveform generation are allowance for micro-tuning and automatic monitoring of the output with feedback adjustment; the advantages provided by DAC waveform generation are smaller physical electronic circuit area and footprint, subsequent lower manufacture cost and ease of integration with other circuits that comprise the complete electronic stimulation device.

In another aspect of the present invention the amplitude modulated waveforms generated by the electronic circuits of the stimulation device are utilized to stimulate and regulate specific intracellular second messengers, including but not limited to cyclic AMP: adenosine 3′,5′-monophosphate (cAMP), and cyclic GMP: guanosine 3′,5′-cyclic monophosphate (cGMP) The chemical pathways, interactions and number of functional processes regulated by these second messengers are very large and extensively studied in the medical-scientific literature and so are not enumerated here. The medical advantage of this utilization is that in many medical instances its inclusion eliminates much of the uncertainty about the appropriateness and predicted effectiveness of the ES, since the functional processes regulated by the second messengers are already known in great detail and activation by them therefore far more predictable. A further medical advantage is that with targeted stimulation of specific second messengers, the ES is essentially, directly boosting what the body is already doing to normalize virtually any pathology at hand, without the human medical physician or technician needing to make extremely complex decisions for the best ES treatment strategy across different timeframes and in relation to the specific nature of the pathology, and further, without risking the disruption nor blocking of normal physiologic healing processes.

In a preferred embodiment of the present invention, the stimulation device can operate as a constant voltage source outputting the DDS or DAC generated amplitude modulated waveforms that results in clinically effective ES at very low peak Output Voltages typically at the lower end of the 1-200 millivolt range and often as low as SOmillivolts, which is far below the levels of Output Voltages of waveform generating technologies and devices in the prior art necessary for them to give lesser or comparative therapeutic effects under the same conditions.

Another aspect of the present invention in relation to intracellular second messenger stimulation is that the software of the stimulation device generates a repeating, timed stimulation cycle that includes an up-regulating component that increases the production of a specific second messenger, followed by the possibility of a first rest period of variable programmable duration, then followed by a component that stimulates the pathway activation of that second messenger, and then again by the option of a second rest period also of programmable variable duration, when the whole second messenger stimulation cycle then repeats from the beginning. The present invention discloses the following base and signal frequencies confirmed during the clinical and laboratory research of the inventors: 4000 Hz modulated by 10 Hz for up-regulating production of cAMP (labeled cAMP_HZ1 in process step ML1-8 of the MULTI TYPE ES WOUND HEALING FLOWCHART shown in FIG. 5), followed by 4000 Hz modulated by 20 Hz for increasing pathway activation and utilization of cAMP (labeled cAMP_HZ2 in process step ML1-8 shown in FIG. 5), and, 4000 Hz modulated by 25 Hz for up-regulating cGMP production (labeled cGMP_HZ1 in process step ML1-9 of FIG. 5) followed by 4000 Hz modulated by 20 Hz for increasing pathway activation and utilization of cGMP (labeled cGMP_HZ2 in process step ML1-9 of FIG. 5). Though it should be understood by those skilled in the art that this aspect of the present invention is not limited to stimulation and regulation of only cAMP and cGMP and that the basic ES principle herewith disclosed can be applied to the stimulation of any number of other second messengers. It should also be understood that utilizing a 4000 Hz base frequency for modulated signal frequency transmission for therapeutic second messenger stimulation is only one possible base frequency within the device's capable 1-20,000 Hz base frequency range and that a key feature of this aspect of the present invention is the ability to match the frequency-dependent (low applied Output Voltage) impedance profile of the internal body tissues lying between the cutaneous electrode pads in which the resultant current is induced to enable accurate and precise internal body signal frequency transmission by means of adjusting the base frequency. Furthermore, the basic logic and biological effectiveness of stimulation of second messengers and especially of cAMP by means of chemical (drug) intervention is well and generally established, whereas the present invention discloses a dedicated electromedical approach that can achieve these same results.

In a preferred embodiment of the present invention, the first rest period following the production component and the second rest period following the pathway activation stimulation component of the stimulation cycle are typically of 1-3 minutes duration in order to maximize the overall biostimulation effect by allowing the electrochemical intracellular second messenger processes to physiologically synchronize with the stimulation cycles, and not be overwhelmed by the electrical energy that is a common pitfall of many ES approaches.

In another preferred embodiment of the present invention, the ES combination switch sets already described include all possible combinations of specific intracellular second messenger stimulations, comprising top level binary switch states of on and off, and binary second-level switch states for production upregulation stimulation followed or not by rest period and pathway activation stimulation. The medical advantage of these second-level switch states is the ability to regulate pathway activation of second messengers such as cAMP that have mediating and controlling effects on various bio electrochemical dependencies such as keratinocyte directional migration under the influence of an electric field as autologously generated by a healing wound having sufficient normal transepithelial electrical potential.

To illustrate the general medical and electrotherapeutic advantages of the intracellular second messenger ES feature of the present invention, various well established beneficial therapeutic effects of cAMP production and pathway activation up-regulation are summarized, as examples only that should be understood do not describe limits to the scope of this aspect of the present invention:
a) Decrease of the overall activity and the specific electrical voltage across nociceptor membrane ion channels, reducing or stopping the transmission (blockade) of mostly neuropathic pain signals, rather than overwhelming the natural pain generating process as with transcutaneous electrical nerve stimulation devices;
b) Blocking smallest diameter, mostly C type fiber motor and sensory nerves, resulting with the blocking of efferent fibers in the vasodilation of arterioles, increased circulation, less perceived pain, local muscle relaxation, increased flushing of toxic metabolites, increased nutrient, enzyme and hormone uptake, and less neurogenic inflammation;
c) Up-regulation of metabolic activity for injury healing including gene expression;
d) Increase in neuron and axon survival and regeneration following injury;
e) Immune and non immune system inflammation modulating effects giving immediate and long-term protection of injured tissues;
f) Reduction of muscle tension and central nervous system modulated anti-spastic action following injury;
g) Increased functional stimulation and regeneration of muscle, and peripheral, and possibly, central, nerve tissue;
h) Enhancement of whole brain and synapse plasticity following injury;
i) Apoptosis stimulation;

In another aspect, the present invention provides a medical iontophoresis system for non-invasive treatment of both surface and deep body bacterial and viral infections via microorganism corresponding discrete low intensity direct electric currents (LIDCs) that pass through the high electrical resistance of intact skin for deep infections, the system comprising:
a control unit with a realtime microcontroller regulated nanoampere output current resolution and accuracy in the ultra-low LIDC range further calibrated by self-adaptive algorithms and full-feedback proportional-integral-derivative control,
a pair of cutaneous electrodes connected to the control unit having extremely high, intact skin interfacing electrical conductivity,
wherein in use:
   the electrodes are positioned on anatomically opposite planar surfaces superficial to the aligned deep body infection target, and
   surface wound healing treatment is performed by creation of an electric field in polarity, strength and topography matched to the individual endogenous wound-generated electric field, derived from direct bioelectric measurement by the control unit and self-adaptively via series resistor algorithmic software wound modeling with resistor voltage drops corresponding to endogenous bioelectric properties of the various types of tissues aligned between the electrode pair.

In another aspect, the present invention provides a medical iontophoresis system for treatment of a body part, the system comprising
a control unit and at least one pair of electrode pads, the electrode pads being for placing on opposing margins or sections of the body part,
wherein the control unit is operable to create a resistance measuring circuit, a current producing circuit, and/or a voltage producing circuit with the electrode pads across the body part,
and dedicated software that implements the operational flowcharts disclosed below that integrates with the control unit in relation to its electronic functional characteristics.

In one embodiment, the control unit is operable to measure resistance across the body part at specified intervals.

In another embodiment, the control unit automatically adjusts the Output Current or output voltage in response to the present measured resistance to provide a substantially constant current or substantially constant voltage.

In another embodiment, the control unit is operable to create a voltage circuit with reversed polarity.

In another embodiment, the electrodes comprise a Positive Electrode and a Return Electrode.

In another embodiment, the electrodes are silver-nylon electrodes.

The present invention also provides a method of non-invasive treatment of both surface and deep body bacterial and viral infections via microorganism corresponding discrete low intensity direct electric currents (LIDCs) that pass through the high electrical resistance of intact skin for deep infections, the method comprising:
providing a control unit with a realtime microcontroller regulated nanoampere output current resolution and accuracy in the ultra-low LIDC range further calibrated by self-adaptive algorithms and full-feedback proportional-integral-derivative control, providing a pair of cutaneous electrodes connected to the control unit having extremely high, intact skin interfacing electrical conductivity,
positioning the electrodes on anatomically opposite planar surfaces superficial to the aligned deep body infection target,
performing surface wound healing treatment by creation of an electric field in polarity, strength and topography matched to the individual endogenous wound-generated electric field, derived from direct bioelectric measurement by the control unit and self-adaptively via series resistor algorithmic software wound modeling with resistor voltage drops corresponding to endogenous bioelectric properties of the various types of tissues aligned between the electrode pair.

The present invention also provides a method of low intensity direct current and medical iontophoresis for treatment of a body part, the method comprising:
placing at least one pair of electrode pads on opposing margins or sections of the body part,
operating a control unit to create a resistance measuring circuit, a current producing circuit, and/or a voltage producing circuit with the electrode pads across the body part.

In one embodiment, the method comprises an Electrode Contact Check Procedure to ensure the electrodes are placed properly on the patient's body.

In another embodiment, the Electrode Contact Check Procedure comprises application of a test voltage across the body part, measuring an average resistance value over a set time interval, and comparison between the measured resistance against a pre-determined test resistance value.

In another embodiment, the method comprises an R Value Calculation Procedure to measure resistance across the body part.

In one variant preferred embodiment (not shown), the R Value Calculation Procedure comprises repeat application of a test voltage across the body part, measuring and recording an average resistance value over a set time interval, and performing real-time updating variance-weighting, algorithmic and statistical analysis of these measurements and various comparisons between these data against pre-determined maximum resistance values and percentage variation limits in relation to the characteristics of the electronic circuits of the control unit 20.

In one embodiment, the method comprises a Sterilization Procedure wherein a constant current is applied across the body part.

In one embodiment, the voltage is automatically adjusted to provide constant current based on the present R value.

In one embodiment, the method comprises a Current of Injury Supplementation Procedure wherein a constant voltage is applied across the body part based on the present R value.

In one embodiment, the voltage level is automatically based on the present R value.

In another embodiment, the voltage is applied with reversed polarity.

In another embodiment, the voltage is between 150 millivolts to 1.1 volts.

In another embodiment, the method comprises a Fibroblast De-Differentiation Stimulation Procedure wherein a constant voltage is applied across the body part.

In one embodiment, the voltage level is automatically based on the present R value or input Positive Electrode surface area.

According to a first aspect, an advantageous feature of the preferred embodiment is that it provides specific and clinically determined combinations of microcurrent intensities (disclosed below) to the silver-nylon (AgN) electrodes (disclosed below) resulting in operational modes for elimination or attenuation of: bacterial infections, viral infections, and promotion and acceleration of wound healing (infected or not), and tissue repair and regeneration.

Another advantageous aspect of the preferred embodiment, is the microcurrent DC stimulator comprising three electronic circuits that are integrated operationally by electronic switching componentry into several operational modes, namely, a resistance measuring circuit, a constant current producing circuit, and a constant voltage producing circuit having polarity switching capability All three circuits, in addition to their absolute operating ranges (0-200 microamperes Output Current, 10 millivolt resolution Output Voltage, 100-3.8E+06 ohms Resistance Measuring) also have a high degree of accuracy of measurement and current and voltage production of these parameters (resolution/accuracy: ±100 nanoamperes, ±10 millivolts, ±10% measured ohms), that are further calibrated for increased accuracy beyond these values by internal algorithms and an on-board full-feedback proportional-integral-derivative (PID) controller that corrects for deviations across wide output and measurement ranges, as well as environmental operating parameters such as temperature and humidity, not available in the prior art.

The preferred embodiment's circuitry can operate reliably and with the identical precision within the extreme temperature operating range of −10 C to +60 C.

Other aspects of the invention are also disclosed.

### BRIEF DESCRIPTION OF THE SEVERAL VIEWS OF THE DRAWINGS

Notwithstanding any other forms which may fall within the scope of the present invention, preferred embodiments of the present invention will now be described, by way of examples only, with reference to the accompanying drawings.

FIG. 1 is a perspective view of a medical Iontophoresis Device in accordance with a preferred embodiment of the present invention.

FIG. 2 is a schematic view, illustrating an electrical stimulation electrode pad according to a first preferred embodiment.

FIG. 3 is a schematic view, illustrating an operational flowchart of the medical iontophoresis system when operated in ‘WOUND’ mode.

FIG. 3a is a schematic view, showing the Electrode Contact Check Procedure of the operational flowchart.

FIG. 3b is a schematic view, showing the R_{wound} Value Calculation Procedure of the operational flowchart.

FIG. 3c is a schematic view, showing the Sterilizing Procedure section and treatment pause section of the operational flowchart.

FIG. 3d is a schematic view, showing the Current of Injury Supplementation section of the operational flowchart.

FIG. 3e is a schematic view, showing the Fibroblast Stimulation section of the operational flowchart and the R_{wound} Value Calculation Procedure of the operational flowchart.

FIG. 3f is schematic view of a table of currently used preferred numerical parameters employed by the algorithms of the flowchart as used during the testing phase of development of the Device.

FIG. 4 is a schematic view, showing the Function Optimization Algorithm Flowchart, labeled the ML (Machine Learning) CONTROL FLOWCHART.

FIG. 5 is a schematic view of a MULTI TYPE ELECTRICAL STIMULATION (ES) WOUND HEALING FLOWCHART, which interconnects with FIG. 4 at step ML2.

FIG. 6 is a schematic view, showing an ES Switch Set Codes Table according to a preferred embodiment of the present invention for a wound healing application.

FIGS. 7A and 7B are schematic views, showing the in vivo complex impedance (Z) sweeps between two SIS AgN electrodes together with simultaneous direct current (DC) resistance measurements made in parallel with the stimulation waveform generation circuit at a low millivolt Output Voltage. FIG. 7A shows the complete frequency range Z sweep and FIG. 7B shows the same Z sweep up to the approximately 5000 Hz range.

### DETAILED DESCRIPTION OF THE INVENTION

It should be noted in the following description that like or the same reference numerals in different embodiments denote the same or similar features.

Structure of the Medical Iontophoresis System

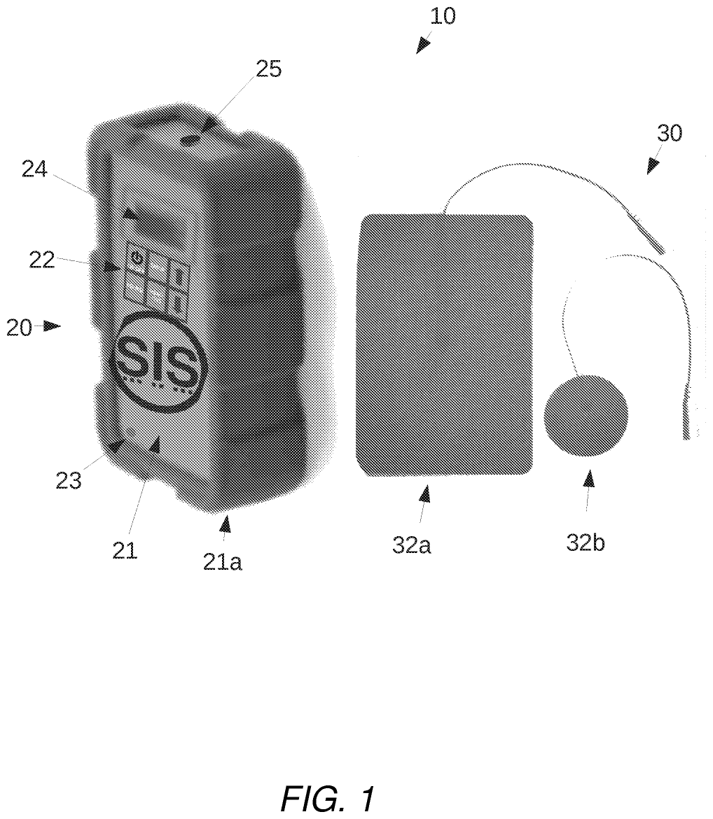

FIG. 1 shows a medical iontophoresis system 10 according to a preferred embodiment of the present invention. Specifically, the example shown relates to a Silver Iontophoresis Stimulator (SIS machine). The system 10 comprises a control unit 20 and at least one pair 30 of electrode pads 32a and 32b The system 10 shown comprises a single pair 30 of electrode pads 32. It is to be understood however that the system 10 can comprise any desired number of pairs 30 of electrode pads 32 and that the electrodes can be of various sizes and shapes.

Briefly, in use, the electrode pads 32 are placed in contact with the patient's body to anatomically cross-section a target anatomical area or location, often an organ, bone, etc. In another example, the electrode pads 32 are placed adjacent to the wound edge and behind the wound on the anatomically opposite surface of the injured body part. The control unit 20 charges the Positive Electrode 32a electrically positive and an electric circuit is completed by the second Return Electrode 32b. Both electrodes are silver (Ag)-nylon (AgN) electrodes 32. A microampere current is produced in the in vivo circuit partly consisting of Ag cations (“Ag particles”, “Ag ions”, “Ag nanoparticles”, “Ag+”, etc) moving between the two electrodes 32 that will thus pass through the tissues anatomically aligned between the two electrodes 32. Extensive international research cited has shown that Ag cations have broad microbicidal, microbe-attenuating (especially bacteria and viruses but also fungi and yeasts), as well as cellular phenotype modifying effects, including inducing de-differentiation of mature fibroblasts that then become pluripotent cells.

The control unit 20 comprises a housing 21 which in one embodiment is IP65-68 rated or IP65-68 performing, meaning it is water-proof and dust-proof, and in another embodiment (shown) is surrounded by a shock-proof silicon covering 21a. At the front surface thereof, the control unit 20 comprises an electromagnetically shielded (membrane) keypad 22, and a rechargeable battery LED charge indicator 23 to indicate battery charge status. The control unit 20 also comprises either an LCD or OLED screen (and in another embodiment not having an LCD or OLED screen and instead LED indicators) that may be integrated within the keypad 22 (not shown). The keypad consists of controls for sound alerts and indications, LCD backlighting, powering on and off the control unit 20, toggling between the operational modes of the control unit (‘BACT’, ‘VIRUS’, ‘WOUND’, ‘REGEN’, ‘WATER’, ‘MICRO1, ‘VOLT’), and additionally, for extremely high-resolution current (±100 nanoamperes) and voltage adjustment, selection of display on the LCD or OLED screen 24 of electrical stimulation and bioelectric parameter values, as well as direct control access to the constant current and voltage switching circuits. The control unit 20 also comprises a connection (jack/socket) 25 for receiving the wire (harness) on the top surface thereof for connection to the electrodes 32, which in all embodiments is of an IP67 water-proof and dust-proof type. The control unit 20 is powered by standard replaceable batteries of the AAA, 9V or AA types contained within a battery compartment on the rear face of the housing 21 and protected by the shock-proof cover 21a in that embodiment.

The control unit 20 provides ‘intelligent’ visual and audio feedback and alerts via the LCD or OLED 24 or LED indicators (not shown) and from an audio unit (not shown) within the housing 21. These feedback means inform the user of the operation of the control unit 20 and direct the user to problems arising in use. The problems can relate to target Output Current or voltage to the Positive Electrode 32a, excessively high resistance values encountered and measured in the entire circuit by the control unit 20, circuit break between the electrodes 32a and 32b, insufficient and/or undesirably fluctuating contact of one or both the electrodes 30 to the body depending on the selected operational mode of the control unit 20, misplacement of the electrodes 32 in cases of application to wounds, and to how to solve these problems independently, if possible.

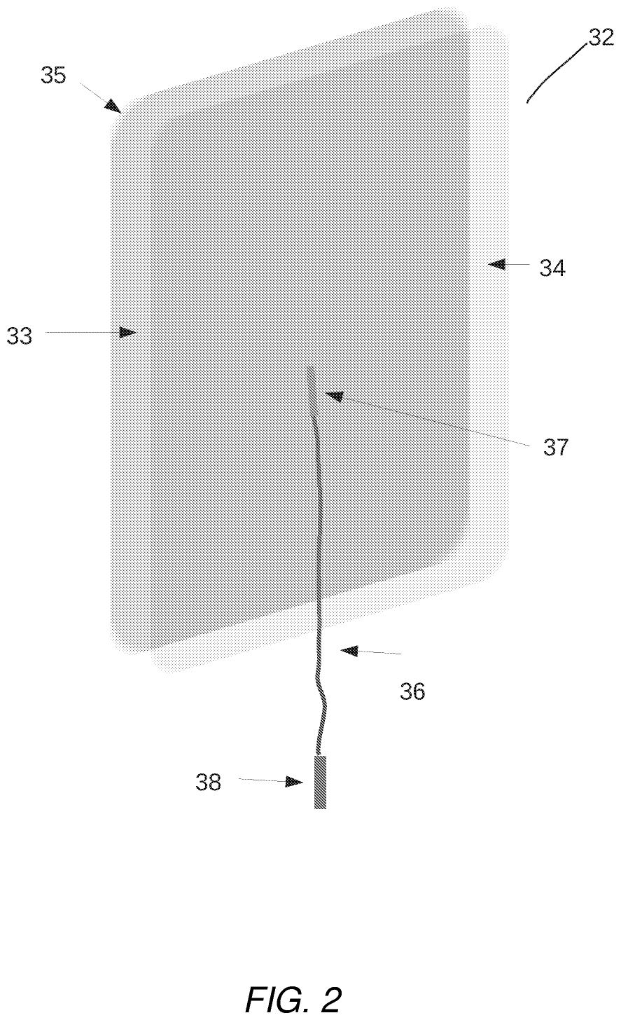

The Positive Electrode pad 32 as shown in FIG. 2 includes an active stimulating surface material 33 comprising pure (9999+%) medical-grade silver (Ag) on a rip-stop and carbon-backed nylon substrate. The active surface 33 is mounted to a medical grade white foam backing 34 having a thickness of 1/32 inches (0.8 mm) and having rounded corners 35 to prevent mechanical skin irritation. In one embodiment (not shown), the white foam backing 34 extends by at least ⅝ inches (1.6 cm) from all edges of the active surface 33, with the extension portions having non conductive skin adhesive material. The electrodes 32a and 32b comprise an extremely low impedance pure copper wire 36 sandwiched between the active surface material 33 and the backing 34, and having its exposed conductive end 37 placed and in contact with a central portion of the active surface 33, and a small amount of medical grade glue (not shown) applied away from the exposed conductive end to secure it in place when in use. The wire 36 is further secured and contact with the silver-nylon (AgN) 33 is ensured by a high conductive adhesive strip running the length of the electrode pad 32. In the embodiment where the foam backing extends from the active surface, the pad 32 can include a cover panel (not shown) which covers a section of the wire 38 adjacent the edge of the backing 34 such that the wire 36 does not contact the patient's skin in use. The cover panel in this embodiment is polyethylene fabric such as that sold under the trade mark TYVEK. The wire 36 comprises an end pin receiving connector 38 for connection with the end pins of a standard electro-stimulator wire such as used with TENS devices, and via this wire to the control unit 20. The second Return Electrode 32b is identical in construction to the electrode 32a as above but as with the Positive Electrode can vary in size and shape or in a much less preferred embodiment can be a normal TENS adhesive or non adhesive electrode. In another preferred embodiment, the connecting wire 36 is constructed of non PVC thermoplastic elastomer having a high tensile, break and corrosion resistant plated internal core, gold-plated connections and an IP68 rated (100% dustproof & waterproof) screw locking connector and socket.

Medical Iontophoresis System—WOUND Operational Mode

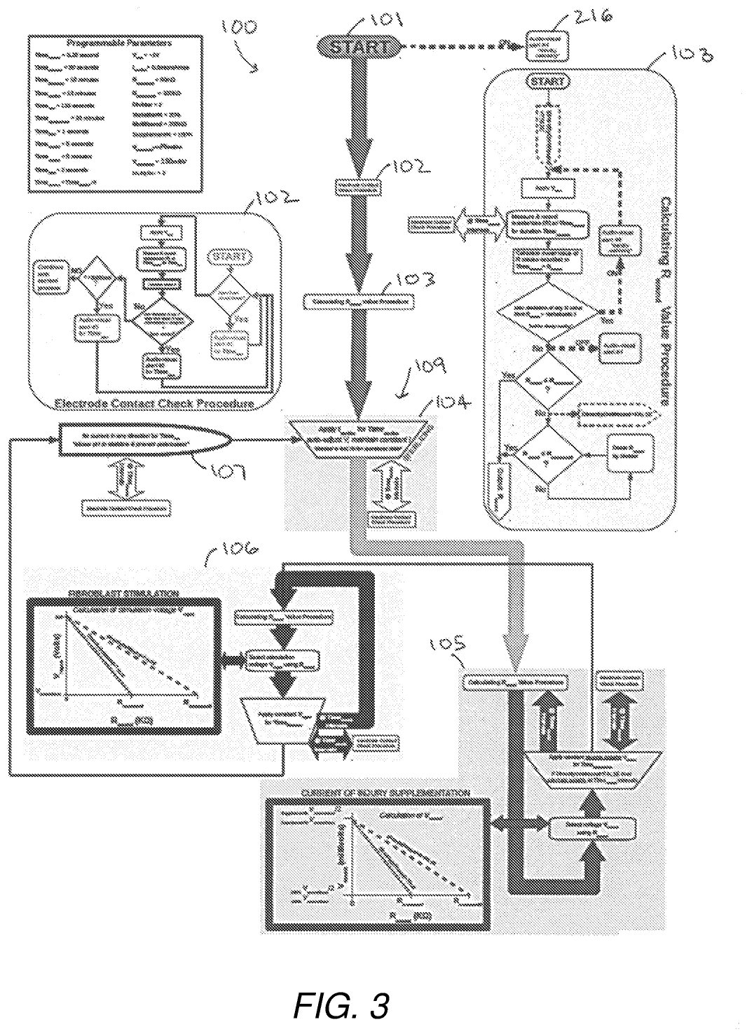

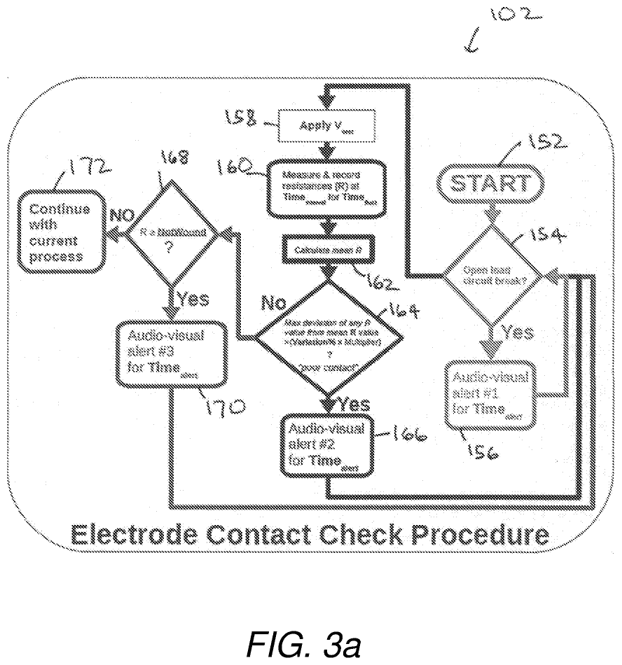

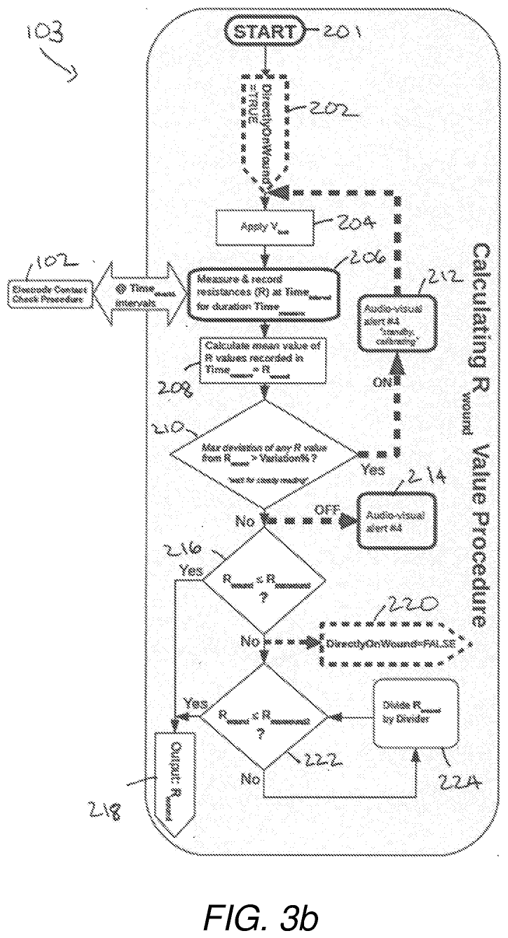

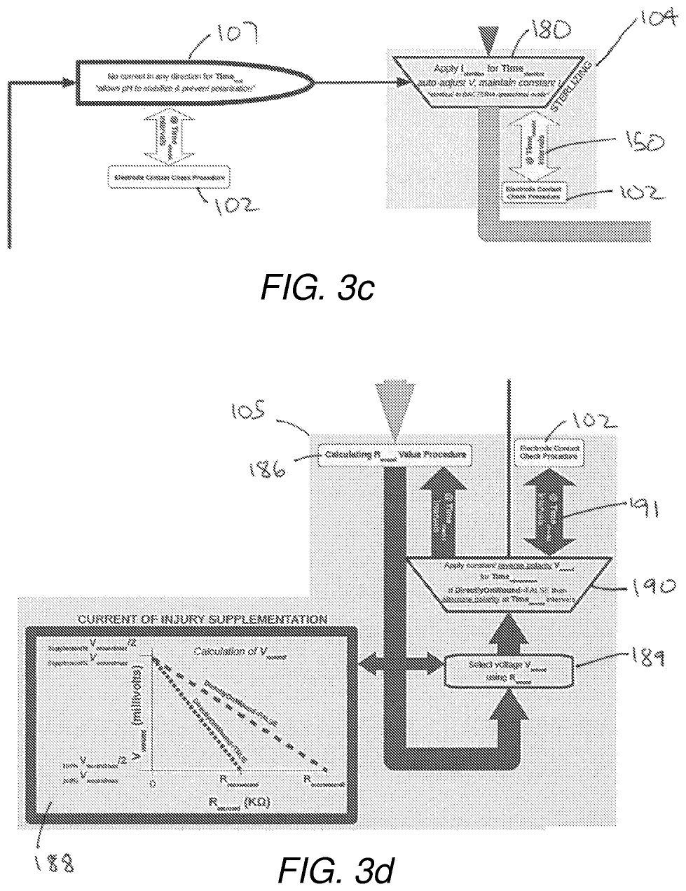

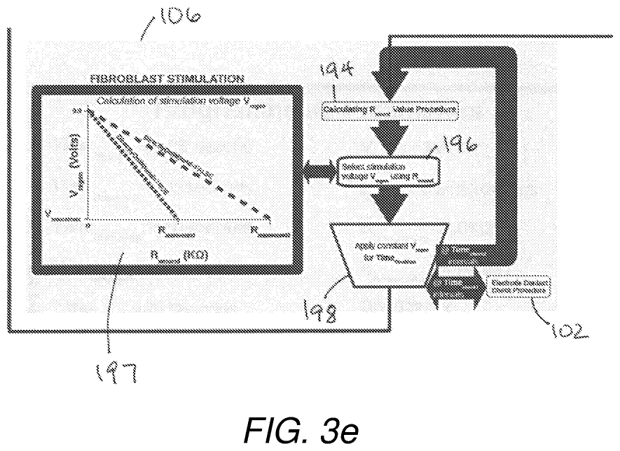

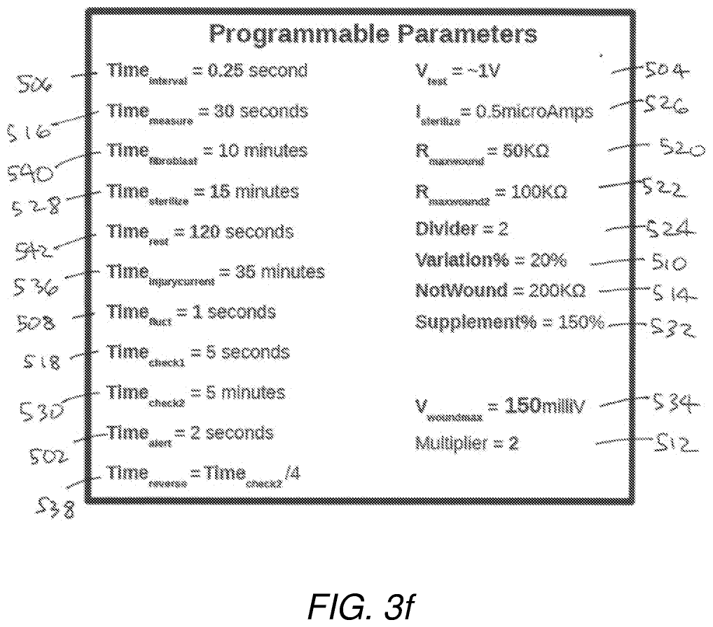

FIG. 3 shows a first mode of operation for treatment of acute or chronic surface wounds, deeper wounds, ulcers, abscesses, other lesions, osteomyelitis, and surgical site infections and protection. The control unit 20 is placed in WOUND operational mode and the two electrodes 32 are connected to the control unit 20.

Application of SIS Electrodes to Wound

Depending on the wound type, there are 3 methods of applying SIS electrodes to wounds.

Method #1: Superficial Wounds (Infected)—The wound is initially irrigated with saline or other sterilizing liquid if available. The Positive Electrode 32a is positioned on the surrounding normal tissue, carefully not to disturb the wound, no more than 2 cm (¾″) from the edge of the wound. The Return Electrode 32b is positioned at an anatomically opposite surface to that of the wound on the same limb if the site of injury is peripheral. On a limb for example, the Return Electrode 32b will be placed on the opposite side of the limb. The Return Electrode 32b is approximately the same size or slightly larger than the Positive Electrode 32a and cut to size if necessary. The Return Electrode 32b is placed onto intact skin as much as possible directly behind the wound on the anatomically opposite surface of the injured body part so that the wound is aligned maximally between the two SIS electrodes 32. This positioning minimizes the chance that silver ion flow from the Positive Electrode 32a will pass through the skin between the two SIS electrodes 32a and 32b instead of penetrating deeper into the wound. If Method #1 electrode positioning is not achievable due to a conventional wound dressing considered not removable at the time of application, then the Positive and Return Electrodes 32a and 32b are positioned across the wound on the same anatomical surface, adjacent opposing margins thereof, on non damaged tissue approximately 10-20 mm from wound margins or as close as possible without causing further stress or damage to the wound. The Positive and Return Electrodes 32 can be placed either way around across the wound

Method #2: Deeper Wounds (Infected)—the wound is initially irrigated with saline or other sterilizing liquid if available. The Positive Electrode 32a is to be placed directly on top of or packed into the wound, and is cut to size so that there will be no or very minimal electrode extending out of the wound in any direction when placed onto the wound bed. The Positive Electrode 32a is rinsed with saline or other sterilizing liquid and placed directly onto or packed directly into the wound. The Positive Electrode 32a is then covered with saline rinsed gauze or other moisture holding dressing if available. The positioning of the Return Electrode 32b is similar to Method #1 as above.

Method #3 First Aid (Positive Electrode only or with Return Electrode and control unit 20 SIS machine)—the wound is initially irrigated with saline or other sterilizing liquid if available. The user then selects a Positive Electrode 32a large enough to cover the entire wound extending at least 2 cm (¾″) beyond wound margins on all sides. The Positive Electrode 32a can also be cut to size if necessary. The Positive Electrode 32a is rinsed with saline or other sterilizing liquid if available and applied directly over the wound. The positioning of the Return Electrode 32b is similar to Method #1 and Method #2 as above.

The SIS electrodes 32 are held onto the skin or wound using adhesive surgical or wound dressing tape (e.g. Fixomull™ or Micropore™), and/or stretch Velcro® strap, bandages or other emergency means as the cover dressing.

Operation of Medical Iontophoresis System for Wounds

FIG. 3 shows an operation flowchart 100 of the system 10 in WOUND mode. Operation starts 101 with the activation of audio-visual alert #4 in block 216 that indicates to the user that the control unit 20 is calibrating to the injured tissue properties and then goes to step 102 where the system performs an Electrode Contact Check Procedure as shown in FIG. 3a. The system then performs step 103 Calculating R_{wound} Value Procedure as shown in FIG. 3b.

The system operation then moves to a treatment loop 109 which comprises a Sterilization Procedure 104, calculation and application of Current of Injury Supplementation Procedure 105, Fibroblast Stimulation Procedure 106, and a rest period 107. The treatment loop 109 then returns to the Sterilization Procedure 104.

The details of the above procedures are described below. The general purpose of each step is as follows:

a) Electrode Contact Check Procedure 102—to ensure the electrodes are placed properly on the patient's body

b) R_{wound} Calculation Procedure 103—to measure resistance of the wound. This is a second stage more sensitive special electrode placement check to ensure stable contact with the wound bed or periwound, and further, differentiates if the electrodes have been placed in or next to the wound.

c) Sterilization Procedure 104—applying constant current across wound to sterilize, with Voltage (V) auto-adjusted for constant current (I). This is to kill and inhibit bacteria and other microbes in the wound, sterilizing the wound by strong antibacterial effect. Electronically it is delivered by what is termed ‘constant current’ circuitry, killing/strongly inhibiting the bacteria.

d) Current of Injury Supplementation 105—applying reverse polarity Vwound, calculated using R_{wound} across the wound. The skin is an efficient electrical battery. As a result of normal, bi-directional ion flow, a constant electrical potential of 10(20)-50(70) millivolts is maintained across the skin layers, termed the transepidermal or transepithelial potential (TEP) difference. When skin layers are damaged in any kind of wound, the collapse of their electrical resistance to the TEP ‘skin battery’, immediately results in an electric field that is electrically negative at the wound edges relative to the deeper tissues. This wound-generated electric field continues until wound closure. Many cells involved in the healing response, having outside positive transmembrane electrical potentials, move outwards to the wound surface under the influence of the wound-generated electrical field These electric field-sensitive cells include osteoblasts, osteoclasts, keratinocytes, neural crest cells, endothelial cells, epithelial cells, chondrocytes, granulocytes, fibroblasts and leukocytes. The process indirectly measures the wound-generated electric field in realtime by skin resistance measurements and known values of the TEP and of the relative voltage potentials at the wound margins, and supplements the wound-generated electric field in a realtime scaled manner to the wound properties so as to generate a bioelectrically matching magnitude and polarity voltage drop at the wound edges.

e) Fibroblast Stimulation Procedure 106—the idea of this stimulation is that it reproduces the method shown in U.S. Pat. No. 5,814,094 (Iontopheretic system for stimulation of tissue healing and regeneration). The method induces fibroblast cells, to de differentiate back from their specialized form, to become cells with some additional pluripotency or multipotency, so that tissue repair and regeneration potentials are greatly increased. In another preferred embodiment, this procedure is also independently accessible by the user in ‘REGEN’ operational mode of the Device where the Output Current auto scales to either the surface injured tissue and applied Positive Electrode size positioned onto that injured tissue or the user can program the surface area of the Positive Electrode size for internal fibrotic tissue targets for auto scaling of the Output Current to the programmed electrode size, in both cases for the same fibroblast de-differentiation producing effect. For internal fibrotic tissue targets, the calculation of the necessary and appropriate Output Current scaling is achieved by algorithmic analysis that also forms part of the disclosure of this invention.

The current preferred parameters shown in the diagrams are listed in FIG. 3f, and are referred with reference numerals starting from 502.

The Electrode Contact Check Procedure 102 as best shown in FIG. 3a starts 152 with a test 154 for an open load circuit break based on a measurement of an extremely high (pre-set) resistance value threshold, indicating electrodes 32 not adhered or incorrectly adhered to the patient, or a physical break or disconnections of the wires, connections, etc, of the stimulating circuit. An open circuit produces step 156 audio-visual alert #1 for time 502 (FIG. 3f), which can comprise LED, LCD or OLED and audio indicators as with all audio-visual alerts of the control unit 20. Steps 154 and 156 are repeated until the circuit is closed. A closed circuit, indicating adherence to the patient of the electrodes 32, will lead to next step 158 where the system 10 applies a test voltage 504 and then to step 160 that measures and records resistance values at pre-set intervals 506 for several seconds pre-set 226 duration 508. Next step 162 is calculation of mean resistance (R) value of the recorded resistance values.

Next step 164 is calculation of deviation of any resistance value from the mean resistance value. Any deviation over a pre-set percentage value 510 that can be varied by the Multiplier value 512 as shown in FIG. 3f indicates poor and/or fluctuating contact of one or both electrodes 32 and produces step 166 audio-visual alert #2, and returns to step 154. No large deviation leads to step 168 where the mean resistance value is compared to a ‘NotWound’ resistance 514 of 200 kiloohm which indicates normal undamaged skin or a distance of the Positive Electrode 32a or of both the Positive and Negative Electrodes 32a and 32b from the wound edges depending on the method of application as described above, considered too great to allow effective operation of the system 10 when applied according to Method #1. Mean resistance greater than ‘NotWound’ resistance 514 produces step 170 audio-visual alert #3 for time 502 indicating that the electrodes 32 are probably not placed on or next to injured tissue, and return to step 154. A mean resistance value less than ‘NotWound’ 154 produces step 172, an indication to proceed with the current process as electrode contact is confirmed.

The R_{wound} Value Calculation Procedure 103 is best shown in FIG. 3b beginning 201 with step 202 which is the setting of a binary variable, DirectlyOnWound, to the condition ‘TRUE’ that determines several other events at other sections of the operation flowchart 100. Next step 204 is the application of a test voltage 504, which in the step 206 is for a specified time 516 during which resistance values are measured and recorded at pre set intervals 506 The arithmetic mean value is calculated immediately after in step 208 During step 206, the Electrode Contact Check Procedure 150 is performed at pre-set intervals 518.

Next step 210 checks the maximum deviation of resistance values from the calculated mean value compared to a pre-programmed percentage value 510, and if the deviation is too great as determined by another pre-programmed variable then directs operation to audio-visual alert #4 activation 212 and then returns to step 204; and if not too great, directs operation to inactivate 214 audio-visual alert #4 and then to step 216 where the mean resistance value calculated in step 206 is compared to a pre-programmed resistance value 520 being the approximate maximum resistance to be encountered through an open wound before the most superficial skin layers are rebuilt as available in the published literature and confirmed in research (not published) by the inventor. If the outcome of step 216 is within these limits, then the stable measured resistance value of the wound is established and recorded as the final output 218 R_{wound} of this procedure 103; if not, then DirectlyOnWound, is set to the condition ‘FALSE’ 220 and the next step 222 makes another comparison of the mean resistance value calculated in step 206 with a further pre-programmed resistance value 522 that is approximately double that encountered through an open wound at approximately 1-2 cm distance from wound margins that has been determined by the inventor during his research, so as to calibrate for electrode 32 placement across rather than on or into a deeper wound If the mean resistance value calculated in step 206 is greater than this pre-programmed value 522, then it is repeatedly divided in the next step 224 by a pre-set numerical value factor 524 until it is, wherefore step 222 is again done and results in the final output R_{wound} 218 and completion of this section of the operational flowchart 100.

Operation then moves to the treatment loop 109, starting with the Sterilizing action 104 as best shown in FIG. 3c wherein in step 180, the constant current circuit maintains 2.5 microamperes (current 526) for time 528 with dynamic circuit resistance encountered through the patient's body. During step 180, the Electrode Contact Check Procedure 102 is repeated 150 at regular intervals 518 to ensure continuity of electrode 30 contact with the body and wounded tissue.

After completing the Sterilizing section 180 operation moves to procedure 105, being the Current of Injury (COI) Supplementation section of the operational flowchart 100 as best shown in 3d. Initially this step leads to the retrieval 186 of a value of the resistance in the circuit via its determination during the R_{wound} Value Calculation Procedure 103. This step 186 is repeated at pre-set intervals 530 throughout this section 184 of the flowchart. After R_{wound} has been assigned, returning to this section 105 of the operational flowchart 100, a calculation of the voltage to be applied step 189 is determined by an equation that scales the voltage to the total resistance measured in the in vivo enclosing circuit that is the sum combined electrical resistances of the following phenomena: the electrodes' 32 size, especially the Positive Electrode 32a in contact with the wound bed or periwound tissue and so also the wound size and depth and its subsequent magnitude-dependent decrease of electrical resistance as a result of the damaged or missing tissue thereof, the resistance of the periwound/adjacent-wound-edge tissue between the Positive Electrode and the wound edge if Method #1 placement has been applied, the much lower internal resistance of the core of the body, and the resistance of the intact skin beneath the Return Electrode where it is placed on the anatomically opposite surface behind the injury. This equation is graphed in one embodiment as shown in step 188 wherein two plots are drawn that represent modifications of the fixed variables 532 and 534 within the equation for variable scaling of the voltage to the wound depending on the mean resistance value measured during the R_{wound} Value Calculation Procedure 103 that determines if the Positive Electrode 32a is placed directly onto or packed into the wound or if is placed on the periwound/adjacent-wound-edge tissue as in Method #1 described above. In another preferred embodiment (not shown) the wound is mathematically modeled and plotted as a circuit consisting of four resistors in series, with relative voltage drops across each of these resistors corresponding to the dynamic physiological phenomena resistance values comprising the total in vivo circuit resistance as described directly above, either algorithmically derived or directly measured, in realtime by the control unit. In Method #2 electrode arrangement, calculated voltages are thereby proportionally adapted to calibrate for the distance of the Positive Electrode 32a to the wound edge so as to still accurately scale the supplementing voltage to the endogenous wound-generated electric field at that distance. The scale and values of the vertical (Y) axis of the graph have been provided by the published literature in the field of electro-chemical wound healing dynamics where the endogenous TEP has been established and shown to produce a voltage drop at the edges of the wound between approximately 50-200 millivolts Step 190 follows wherein the scaled voltage is applied in reverse polarity for time 536 to supplement the endogenous wound-generated electric field from the inside to the outside of the wound either when the Positive Electrode 32a is placed directly onto or into the wound or onto the periwound/adjacent-wound-edge tissue. Timing and magnitude of all these inter-related events, so as not to negate, diminish, interrupt, or otherwise interfere with the endogenous wound-generated electric field, an advantageous feature for the correct and therapeutically useful operation of each section of the entire flowchart 100, is set by an array of pre-programmed parameters as best shown in 3f. The last action block 191 is that the Electrode Contact Check Procedure 102 is repeated at regular pre-set intervals 518 to ensure continuity of the electrodes' 30 contact with the body and wounded tissue.

Next step 106 is the Fibroblast Stimulation section of the operational flowchart 100 as best shown in 3e. Initially this step leads to the retrieval 194 of a value of the resistance in the circuit via its determination during the R_{wound} Value Calculation Procedure 103. As shown, step 194 is updated regularly during this section 106 of the operational flowchart 100 at pre-set intervals 530. After R_{wound} has been assigned, returning to this section of the operational flowchart 100, the next step 196 is a calculation of the voltage to be applied that is determined by an equation that scales the voltage to the total resistance measured in the in vivo enclosing circuit that is the sum combined resistance of the physiological and electrode elements in the entire circuit as already described above in the Current of Injury Supplementation section 105 of the operational flowchart 100. Here, the scale and values of the vertical (Y) axis of the graph have been provided in the published literature by Becker et al [U.S. Pat. No. 4,528,265 A 1982, U.S. Pat. No. 5,814,094 A 1996] in the field of cell modification by silver iontophoresis. The voltage scaling is adjusted for electrode placement either onto or into the wound or onto the periwound/adjacent-wound-edge tissue of a superficial wound as determined during the repeatedly performed R_{wound} Value Calculation Procedure 103 by means of a variable belonging to the scaling equation in section 106 as shown in the two graphs 197 therein. Once the stimulating voltage has been calculated the next step 198 applies this voltage for a pre set time interval 540 critically determined to integrate with the other sections (3a, 3b, 3c, 3d) of the operational flowchart 100 by the programmable parameters as best shown in 3f. During this entire section of the operational flowchart 100, the Electrode Contact Check Procedure 102 is repeated 199 at regular intervals 518 to ensure continuity of electrode 30 contact with the body and wounded tissue.

In another embodiment of the present invention, the Fibroblast Stimulation section 106 of the operational flowchart 100 as best shown in 3e can be activated separately while still utilizing the Rwo d Value Calculation Procedure 200 and the Electrode Contact Check Procedure 150. In this ‘REGEN’ (regeneration) operational mode available for the expert clinician or researcher the system 10 is adapted for inducing cellular modification of wounded tissue (as described above) as clinical or research needs or aims arise. In this embodiment, when the control unit 20 encounters a skin resistance greater than that of a wound, meaning that the user is targeting internal fibrotic tissue, the appropriate constant stimulation current is determined by a pre-programmed scaling of the current to the electrode size (not shown), which is input by the user via the keypad 28, in order to maintain a proportional current density for the cellular modification effect according to and as already described above in mention of the inventions of Becker et al.

The flowchart 100 then moves to a rest period 107, before returning to the Sterilizing section 104 of the flowchart after completion of the other sections of the treatment loop 109. The rest period 107 is where no voltage is produced to minimize skin irritation from the electrodes 32, electrolysis, and interference with pH dynamics, as well as to prevent cellular polarization, especially overextended use periods, and lasts for a predetermined time 542. During step 107, the Electrode Contact Check Procedure 102 is repeated at regular intervals 518 to ensure continuity of electrode 30 contact with the body and wounded tissue.

Operation of Medical Iontophoresis System for Internal and Surface Infections

The ‘BACT’ (bacteria), ‘VIRUS’ and ‘MICRO’ (microcurrent) operational modes of the system 10 again utilize the constant current producing circuit (disclosed above). In this application the control unit 20 maintains constant low intensity direct currents of 2 5 and 7.5 microamperes respectively for bacterial and viral infections with dynamic circuit resistance encountered through the patient's body, that have been found clinically by the inventor and confirmed by conventional medical pathology laboratory testing to be highly effective in vivo for these microenvironmental infections. ‘MICRO’ operational mode is user-programmable, and so can generate any of these effective Output Currents.

Positive and Return Electrode placement for internal infections is on intact skin such that the target internal infected organ or tissue is aligned as much as possible between the two electrodes. The Positive Electrode must completely ‘cover’ the target internal organ or tissue such that it is at least the same size or slightly larger than the target internal organ or tissue as it would be seen two dimensionally in an X-ray taken from the position of the electrode on the body surface. The Return Electrode must be approximately the same size or larger than the Positive Electrode and then positioned onto the anatomically opposite surface of the body to the Positive Electrode. This electrode positioning configuration focuses silver ion flow into the target organ or tissue between the two electrodes so that ‘wasted’ current flow through the skin between the electrodes is thereby prevented or minimized. Positive and Return Electrode placement for surface infections is on intact skin across the target infected area on opposite sides thereof.

The Programmable Parameters best shown in FIG. 3f that are used throughout the entire operational flowchart 100 and the construction itself of the entire operational flowchart 100 allowing these parameters to be flexibly incorporated is another advantageous feature of the preferred embodiment. As shown, these parameters are the currently nominated preferred parameters used during the testing phase of development of the Device. According to the present invention, these parameters can be readily modified individually or together through wide inter-related ranges as necessary or desired for improved and adapted future system 10 functionality with accumulating clinical experience, range and type of applications on both humans and non-human animals, extreme or very different external environment operating conditions including extremes of temperature and such as might be encountered in remote areas or during emergency natural or man-made disaster situations that may affect internal and skin tissue electro-chemical properties, and for additional extra whole system 10 functionality if new choices and constructions of other electrode 32 conductive materials are employed such as copper (Cu) for treatment of fungal infections, gold (Au), etc.

It should also be noted that another key feature of the preferred embodiment, is that the system 10 is not bound by the exact Output Currents for the ‘VIRUS’ and ‘BACT’ operational modes, that, though clinically determined in the context of the system's 10 research, testing and development, are also parameters that can be varied within the microcurrent range capable by the constant current circuitry, with future improved knowledge and wider clinical experience, and various applications and settings described, within the deliberate design and electronic componentry of the control unit 20 that has been specifically invented in the appropriate form for these purposes.

In another embodiment of the present invention the Device serves as a programmable, self-adaptive, high accuracy and resolution constant low intensity direct current (LIDC) or constant low voltage stimulator, that connects directly via an electrode wire (harness) to a temporary or embedded silver needle anode and second inserted or surface (needle) cathode, for an alternative treatment of acute or chronic osteomyelitis.

In a variant preferred embodiment of the present invention, the Device can continuously assess and confirm surface wound healing (rate), or lack of healing, via realtime measurements and read-outs of changing electrical resistance of the wound surface with granulation tissue formation and rebuilding of skin layers. This assessment has the advantage that it can be performed without having to remove dressings and without visual examination. These data also give information on the relative wetness or dryness of the wound surface which is also an established factor in wound healing.

The preferred embodiment provides a dedicated silver (Ag)-nylon (AgN) electrode and portable low intensity direct current (LIDC) and Iontophoresis Electro-Stimulator integrated system with constant (ultra-low) microcurrent control. The Device is portable for use inside a medical facility, and outdoors for accident, emergency and preventative applications, being powered by replaceable internal batteries that can also be of the rechargeable type. The Device can be used on humans and non-human animals.

The Device of the preferred embodiment provides extremely high accuracy and resolution output (stimulation) microcurrent control/regulation via realtime microcontroller regulated resistance measuring circuitry that measures the resistance in the entire circuit between the Positive and the Return SIS electrodes, constant current circuitry, and voltage producing and switching circuitry, and the related continuous, responsive microcontroller regulated voltage adjustments and operation. The constant microcurrent circuitry range is 0 microamperes to 200 microamperes with stability and accuracy of ±100 nanoamperes, maintained across a wide temperature range also due to the PID system already mentioned above. These features also accommodate changes in body/limb/neck position, tissue hydration changes, other tissue conductivity changes (e.g. sweating), swelling, exudate, and SIS electrode contact decrease and/or variability

The adjustable interval-step of the Output Current is <100 nanoamperes, that is achieved by the user via the membrane keypad 28 in the preferred embodiment; and at any future time variable via firmware updates. The system 10 provides constant Output Current, voltage and circuit break monitoring.

The system 10 also provides intelligent visual and audio feedback via an OLED LCD or other type of electronic display and/or LEDs integrated into the keypad 22 depending on the particular embodiment of the casing of the control unit 20 and audible alerts that inform and direct the user to problems arising with Output Current, voltage and circuit break, and to how to solve these problems independently, if possible.

The incorporated OLED in one embodiment for the expert user allows realtime readouts of voltage, current and resistance while the control unit 20 is in use and/or being arranged/calibrated on the body.

The control unit 20 is also capable of receiving future firmware updates to allow for easy and rapid improved programmable function as new research and clinical findings might reveal.

The novel firmware and software of the control unit 20, in addition to enabling all of the above functionalities, allows two levels of user expertise for broad and general application: ‘non-expert’ and ‘expert1. The non-expert user mode is achieved by a preprogrammed firmware code that automatically adjusts the control unit 20 to deliver clinically tested, viable currents via the correspondingly required voltages for antibacterial effect or for anti-viral effect as selected by the non-expert user. This non-expert user mode is quickly and easily accessible and these Output Currents selectable via the membrane keypad on the exterior of the housing 21 in the preferred embodiment. The expert use mode is achieved by the functionality described above that is made readily programmable by the user and that enables the expert user to select and adjust the control unit's 20 electrical output parameters including the Output Voltage in one variant embodiment and also to view and monitor these stimulation and endogenous bioelectric parameters (circuit resistance, current and voltage). This functionality has broad clinical advantages and applications, for example but not limited to, cases of unusual or difficult anatomical placement of electrodes 32, especially sensitive skin areas, cases of oedema and ascites where higher currents might be necessary, wound healing monitoring, acute and emergency conditions, complex clinical conditions as they change and are monitored frequently with laboratory tests or visual examination over time, for example a case of mixed viral and bacterial infections.

The Device has battery voltage increasing or decreasing buck-boost converter circuitry to supply a voltage below or beyond the maximum 4.5 volts (1.5 volts×3) and 6 volts (1.5 volts×4) of the three and four (rechargeable) AAA size batteries in the various casing embodiments, if needed for example due to exceptionally high skin resistance encountered. This feature is also under dynamic firmware control and adjustment.

In all user operational modes described, the control unit 20 has pre-programmed 226 firmware controlled, several-second to two minute interval complete stops in output voltage 182 for example as shown in FIG. 3b, to minimize or prevent skin irritation, electrolysis, interference with natural pH dynamics, as well as to not ‘overwhelm’ endogenous bioelectric events especially during the Wound operational mode and to also prevent cellular and cellular population polarization, especially over extended use periods.

The preferred embodiment of the Device of the present invention provides the following advantages:

(1) ‘BACT’, ‘VIRUS’ and ‘MICRO’ operational modes Preprogrammed or user-programmable, clinically determined current-to-electrode calibrated settings for effective bacterial and viral infection treatments.

(2) Palm size and simple to use: Positioning electrodes on or across an affected area and push-button operation.

(3) Electrode-skin interface monitoring and user interface: Self-Adaptive Monitoring Device as well as a low intensity direct current to milliampere Electro-Stimulator. Intelligent statistical and algorithmic software constantly monitors the electrode-tissue interface (area). Monitoring is specific to the electronic circuitry, self-adaptive to the target stimulation Output Current, and in relation to programmed, known biological electro-chemical properties. Audio-visual alerts are generated by the software for the user to maintain optimal electrode application for continuous target stimulation Output Current and voltage delivery. Intelligent interfacing is via organic light emitting diode (OLED) LCD display or keypad-integrated light emitting diode (LED) indicators.

(4) Self-adaptive temperature circuit calibration and extreme operating range: Accurate target low intensity direct current (LIDC) delivery with changing ambient (and body) temperature by full feedback proportional-integral-derivative (PID) industrial-type, realtime circuit calibration and control.

(5) Electrodes manufactured from medical grade 99+% silver-nylon and all composite and backing materials, with various sizes.

(6) High accuracy, self-adaptive target constant current delivery: State of the art sensing technology and microcontroller automatically regulates the target stimulation low intensity direct current with environmental changes including (human) animal body hydration, perspiration, position and movement and during in vitro use. Output Current accuracy is a stable ±100 nanoamperes (nA) between 1-20 microamperes (uA) necessary for silver iontophoresis via silver-nylon cloth electrodes.

(7) ‘WOUND’ and ‘REGEN’ (tissue regeneration) operational modes realtime measurement of the electrical resistance of the wound, directly at the wound bed, or calibrated to the adjacent-wound-edge tissue for peri-wound electrode positioning realtime, self-adaptive calculation and scaled voltage supplementation or replacement of the endogenous wound-generated electric field, for bioelectrically matching magnitude and polarity voltage drop generation (simulation) at the wound edges. Automatic calibration of stimulation voltage to electrode placement on the wound bed for deeper wounds, or on the periwound/adjacent-wound-edge tissue for superficial wounds. Output Current also auto scales to wound and electrode size for surface injuries, or user can program the Positive Electrode size for internal fibrotic tissue targets for auto-scaling of current to programmed electrode size.

(8) Very robust IP65-68 (waterproof and dustproof) rated and mechanical stress-resistant casing including all external ports. Designed for indoor and extreme outdoor environments.

(9) Expert (‘Practitioner’) version with high resolution manual current adjustment and realtime integrated display readout of bioelectrical and stimulation parameters.

(10) Hardware and firmware platform designed for easy and fast updates if future research improves electrical stimulation parameters and/or electrode specifications or reveals further therapeutic applications.

The preferred embodiment provides a microprocessor controlled, Iontophoresis Electro-Stimulator electronically designed and firmware controlled to provide appropriate (low and ultra-low) voltages and currents to a silver-nylon (AgN) cloth skin-contacting electrode to charge that electrode electrically positive (anodal) so that when it is placed in contact with the human body and an electric circuit completed by a second, identical electrode also placed in contact with the same human body—anatomically cross-sectioning a target anatomical area/location, a low intensity direct current is produced in the circuit partly consisting of silver (Ag) cations (“Ag particles”, “Ag ions”, “Ag nanoparticles”, “Ag+s”, etc) of nanometer dimension moving between the two electrodes, that will thus pass through the tissues anatomically aligned between the two electrodes. Extensive international research has shown that Ag cations have broad microbicidal, microbe-attenuating (especially bacteria but also viruses and other microorganisms) and/or biological ‘microenvironment’ modifying effects. The Device addresses this specification and medical need.

The preferred embodiment thus provides a dedicated, portable Device to provide appropriately low, high resolution and accuracy milli-range voltages and constant current circuitry to produce highly precise nanoampere to milliampere range currents, to a silver-nylon (AgN) based electrode.

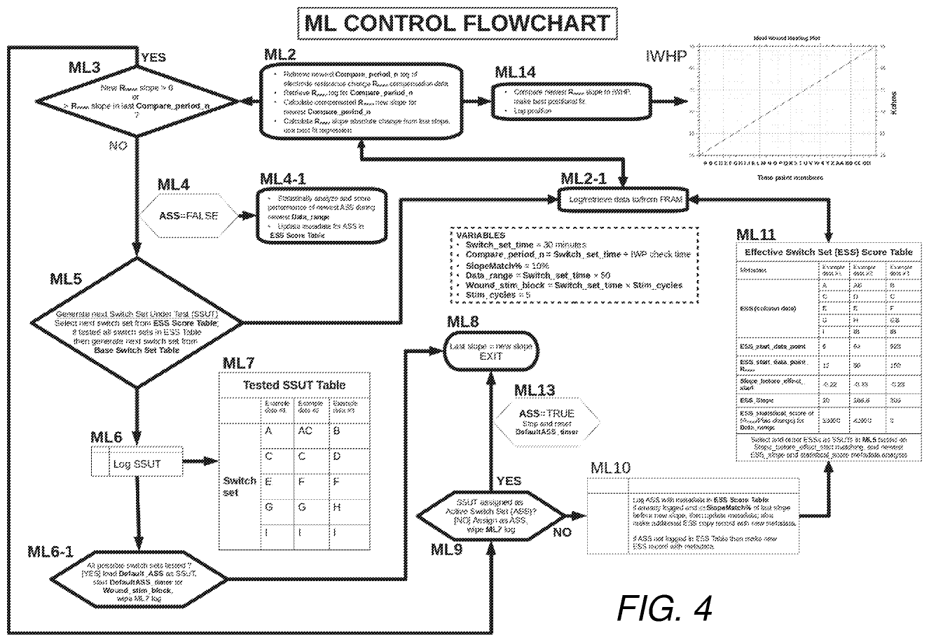

### Brief Description of FIGS. 4 to 6

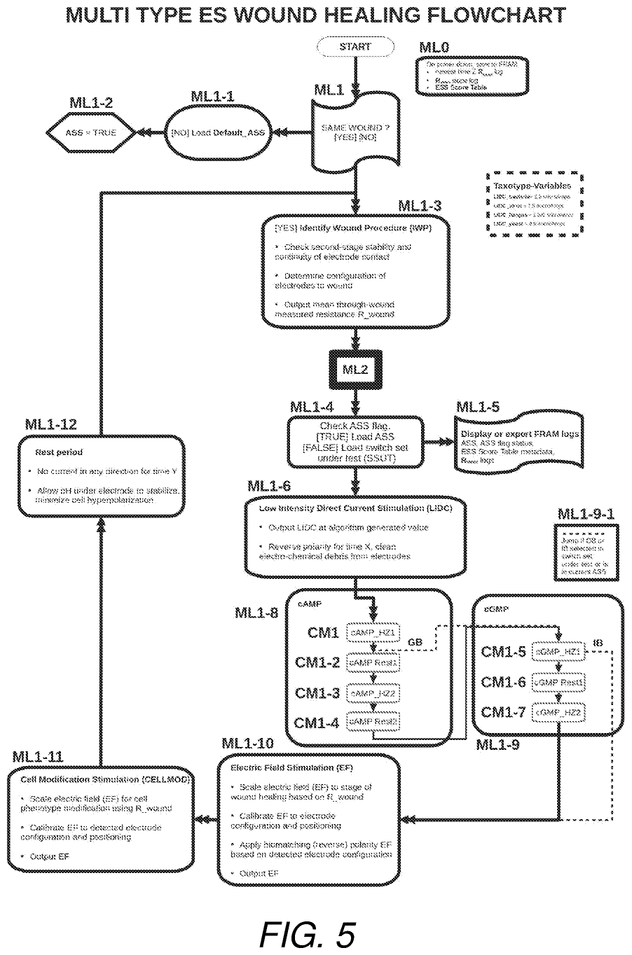

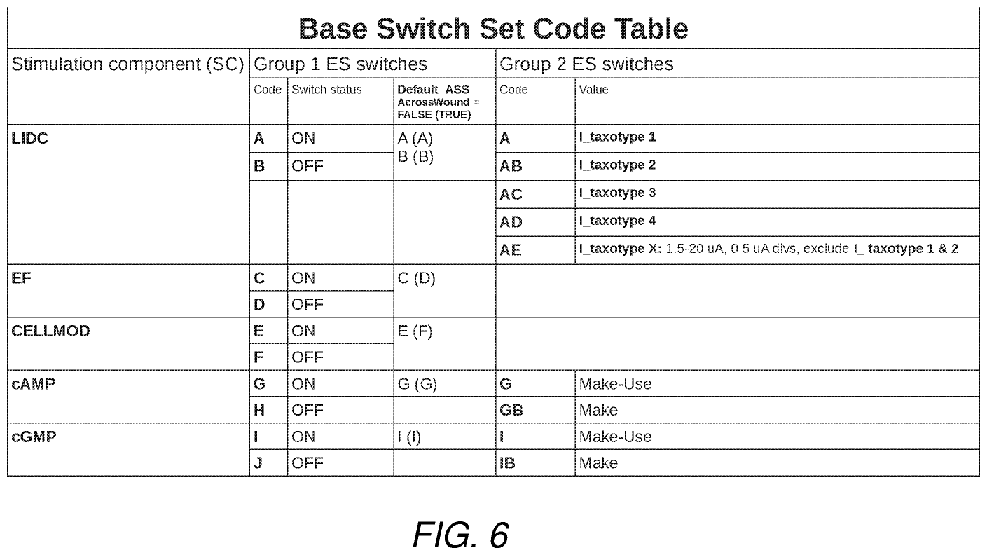

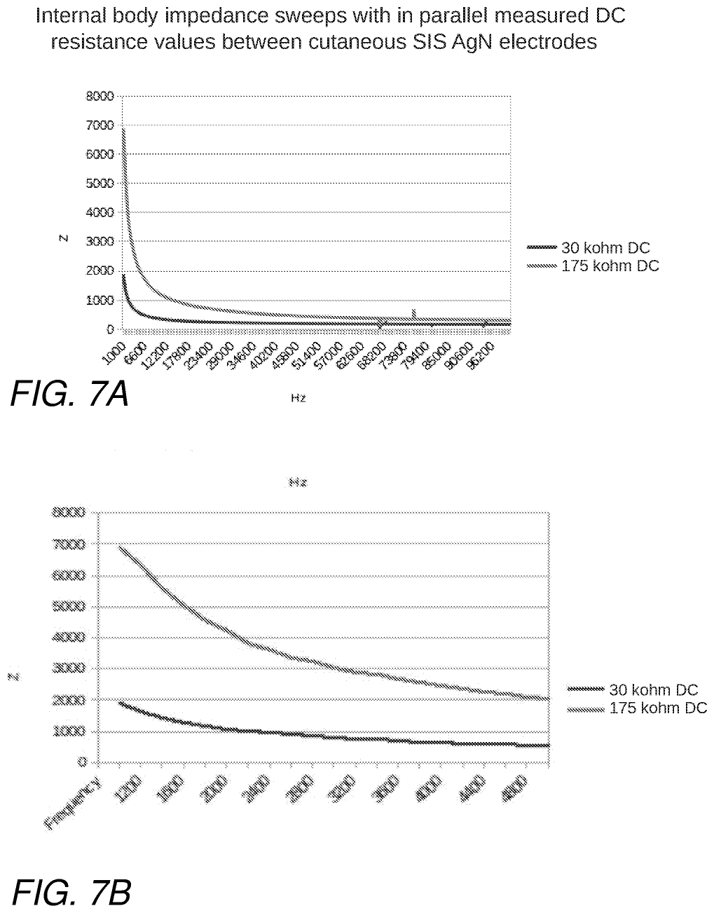

FIG. 4 best shows the function optimization task algorithm flowchart, labeled, ML (Machine Learning) CONTROL FLOWCHART, specifically applied to surface wound healing, which interconnects at step ML2 with the MULTI TYPE ELECTRICAL STIMULATION (ES) WOUND HEALING FLOWCHART as best shown in FIG. 5. FIG. 6 shows an ES Base Switch Set Codes Table according to a preferred embodiment of the present invention, specifically for a surface wound or ulcer healing application.

The MULTI TYPE ES WOUND HEALING FLOWCHART (FIG. 5), is the highest level flowchart of overall operation. The ML CONTROL FLOWCHART (FIG. 4) is a nested sub-procedure of, and ‘called’ from within, the MULTI TYPE ES WOUND HEALING FLOWCHART.

The ML CONTROL FLOWCHART (FIG. 4) is ‘called’ at box ML2 from the MULTI TYPE ES WOUND HEALING FLOWCHART (FIG. 5), after which, process flow jumps out and continues within the ML CONTROL FLOWCHART (FIG. 4), until it exits for any reason at step ML8 of FIG. 4; at which point process flow jumps back and resumes within the MULTI TYPE ES WOUND HEALING FLOWCHART from step ML1-4 (FIG. 5).

At the end of a cycle through the MULTI TYPE ES WOUND HEALING FLOWCHART returning back to ML1-3, box ML2 will again be ‘called’; and this entire process repeats until the device automatically powers off due to uncorrected application error alert, battery charge drainage, or manual control by the user.

The details of the above flowchart procedures are described below. The general purpose of each step is as follows:

FIG. 5—Multi Type ES Wound Healing Flowchart:

Processes within steps ML1-3 (103), ML1-6 (109), ML1-10 (105), ML1-11 (106) and ML1-12 (107) have largely been disclosed in AU patent 2016202751 and further in the specification above; their correspondences within the FIG. 3 flowcharts are indicated in parentheses. These are further described below.

The following two new process steps and all their contents relate to the treatment device's new stimulation capabilities and features: ML1-8, ML1-9. These flowchart steps correspond to the specifications in the Second Messenger Stimulation specifications above.

The following new steps and all their contents relate to the function optimization algorithm shown in FIG. 4 in its entirety: ML0, ML1, ML1-1, ML1-2, ML1-4, ML-2, ML1-9-1.

Steps ML1, ML1-1, ML1-2 obtain from the user or automatically the information if the wound the device and pads are now applied to is the same wound as treated in the previous stimulation session or if the device is now applied to a new wound. For the same wound, the log of Rmean values, the ESS Score Table shown in ML-11 of FIG. 4 and the last active switch set (ASS) are loaded from the stimulation device's ferroelectric RAM (FRAM) memory; for a different wound, the Rmean log is cleared and the Default_ASS is loaded. In all cases, the ASS software flag is set to its positive binary status of TRUE.

Next step Identify Wound Procedure (IWP) as best shown in process step ML1-3, performs a second stage stability and continuity of electrode contact check, and refers to an Electrode Stimulation Efficiency (ESE) module of the software that performs the self-adaptive, first stage, realtime and statistical complex electrode contact check by means of periodically making multiple short timeframe (typical 1-2 seconds) very short interval (typically 200-300 milliseconds) R_wound measurements and logs, and realtime and statistical analysis of these data that translates and assesses the dynamic electro mechanical properties and contact of the electrode pads with the body during each treatment session. The IWP secondarily utilizes the data from the second stage check to determine the location and proximity of the pair of electrode pads to the wound bed based on reference to published data of bioelectric wound measurement and dynamic wound modeling The IWP outputs an arithmetic mean value of multiple through-wound electrical resistance measurements (R_wound), designated, Rmean-(163) The next step as best shown by the interconnecting point to the ML CONTROL FLOWCHART, ML2, is when the Rmean value output from step ML1-3 is exported into the ML CONTROL FLOWCHART (FIG. 4).

Next step as best shown in ML1-4 checks the status of the ASS binary software flag, and loads the current ASS if the flag's status is TRUE, or loads the next switch set under test (SSUT) generated from ML CONTROL FLOWCHART step ML5 if the flag's status is FALSE.

The next horizontal process step as best shown in ML1-5, describes the data that is retrieved from the stimulation device, either via its integrated display or via a data port whereby the device's FRAM can be transferred via a wired or wireless connection for display and analysis on another generic or dedicated electronic display unit. In one preferred embodiment of the present invention as shown in ML1-5, the current SSUT or ASS, the ASS flag status, the Default ASS flag status and any number of previous ASSs and their metadata stored in the ML CONTROL FLOWCHART ESS Score Table shown in ML11 are accessible and can be continuously retrieved in realtime.

Operation then moves to low intensity direct current (LIDC) stimulation as best shown in process step ML1-6 and as also included in a more basic form in AU patent 2016202751. In the present invention, LIDC stimulation has the additional operation, via its interconnection and control by the ML CONTROL FLOWCHART step ML5, of producing an LIDC output value that in a preferred embodiment has multiple second-level switch states as shown in the Base Switch Set Codes Table in FIG. 6, under the Group 2 ES switches column for the LIDC stimulation component, where the values of I_taxotype 1-4 can be pre-programmed Output Current values for various known microorganism taxonomic specific effect-relationships and I_taxotype X can have a range of Output Current values from at least 1.5-200 microamperes with increments of 0.5-10 microamperes or less for automated predictive searching by the function optimization algorithm in step ML5.

The Taxotype-Variables box shows typical default values for the LIDCs that often correspond to major microorganism taxonomies.

Additionally, in another aspect of the present invention, during LIDC stimulation step ML1-6, the LIDC polarity is periodically reversed for 10-30 seconds to clean electrochemical debris from the electrode pads.

Operation then moves to cAMP and cGMP second messenger stimulation as best shown in process steps ML1-8 and ML1-9. Generally, the flow of second messenger stimulation begins with cAMP production stimulation in step CM1, followed by a rest period in step CM1-2, after which cAMP activation stimulation is performed in step CM1-3, with a second rest period in step CM 1-4. cGMP second messenger production stimulation follows immediately in step CM1-5, followed by a rest period in step CM1-6, after which cGMP activation stimulation is performed in step CM1-7.

The dotted intra-process lines between process steps ML1-8 and ML1-9 and between process steps ML1-9 and ML1-10, labeled GB and IB, respectively, and as noted in ML1-9-1, show the flow of processes within and following the cAMP and cGMP second messenger stimulations, when either or both of the second-level switch states for the cAMP and cGMP stimulation components shown in the Group 2 ES switches in the Base Switch Set Codes Table (FIG. 6) are in the binary, Make state of increased production stimulation without a following rest period (step CM1-2 for cAMP, step CM1-6 for cGMP) and activation stimulation (step CM1-3 for cAMP, step CM1-7 for cGMP). Conversely, when either or both of these second-level switch states are in the second binary, Make-Use state, then the rest periods and activation stimulation components are active and so the intra-process lines GB and IB are not followed.

The typical, relative values for the durations of the production components, in between rest periods and activation components for the examples of cAMP and cGMP second messenger stimulation are disclosed above in the Second Messenger Stimulation sections. For various therapeutic applications, the lengths of these durations can be altogether adjusted in relation to Pbio and its biologically possible rate of variability as described above in the Summary of the Invention, and to the measurement resolution capability of the stimulation device to detect these Pbio changes; however, keeping the relative proportions of these durations is a preferred aspect of the present invention. The frequencies of cAMP_HZ1, cAMP_HZ2, cGMP HZ1, cGMP_HZ2 shown in process steps ML1-8 and ML1-9 have also been disclosed under the sections above.

Operation then moves to Electric Field Stimulation (EF) stimulation as best shown in process step ML1-10 and as also included in AU patent 2016202751. An EF is output in biomatching polarity and strength to the wound based on realtime Rmean measurements and on the resultant output value of the dynamic wound modeling using Rmean. The strength of the output EF is further calibrated to the electrode pad configuration and positioning, either paired to a periwound location and anatomically behind the wound, or paired across the wound on either side thereof on the same anatomical surface.

Operation next moves to Cell Modification Stimulation (CELLMOD) stimulation as best shown in process step ML1-11 that is also included in AU patent 2016202751. An EF is output that is scaled to the wound's current bioelectric properties and the (+)positive electrode size for cell phenotype modification based on data in U.S. Pat. No. 4,528,265 A 1982 and U.S. Pat. No. 5,814,094 A, 1996. The output EF is further calibrated to the electrode pad configuration and positioning as also performed in ML-10.

The last stage of a stimulation cycle after completing output of a SSUT or ASS, before repeating, is a rest period as best shown in step ML1-12. No current is output for a specified time Y, typical values of which are shown in FIG. 3f, for the effects of allowing the pH of the skin under the electrode pads to equilibriate and stabilize, and to minimize hyperpolarization of cells influenced by the EF and LIDC stimulations. Flow then returns to step ML1-3 on completion of the stimulation cycle. It should be understood that process flow is either continuous and sequential, or comprises skipped steps, including and between process steps ML1-6 and ML1-12, depending on the selection of the ASS determined by the function optimization task algorithm for each stimulation cycle or multiple cycle durations.

FIG. 4—Function Optimization Task Algorithm Flowchart, Labeled, ML Control Flowchart

In the example application shown of the function optimization algorithm, Rwo is the arithmetic mean value, Rmean, of multiple measurements of the through-wound electrical resistance, R_wound.

The VARIABLES box in FIG. 4 shows the typical values and limits for six of the parameters utilized by the function optimization algorithm for a wound healing application in a preferred embodiment of the present invention. The Switch_set_time,

Compare_period_n and Wound_stim_block_variables are based on the possible biological dynamic rate of change of the through-wound electrical resistance measurement (Pbio) and the measurement resolution, after calibration for electronic circuit temperature effects, of the stimulation device, which are all aspects of the present invention. The IWP check time denominator used to define Compare_period_n is the interval that Identify Wound Procedure (IWP) as shown in process step ML1-3 is ‘called’ within steps ML1-6 to ML1-11 of the MULTI TYPE ES WOUND HEALING FLOWCHART (FIG. 5) and corresponds to Time_check2 as shown in FIG. 3f; and where process step ML1-3 itself largely corresponds to the Calculating R_{wound} Value Procedure as shown in FIG. 3.

ML CONTROL FLOWCHART (FIG. 4) operation starts with electrode pad drying compensation calibration data being imported from the FRAM logs as best shown in and between ML2-1 and ML2, which show a preferred embodiment of this aspect of the wound healing application of the function optimization algorithm. These electrode pad drying compensation data are multiple additional Rmean measurements made and logged periodically within each stimulation cycle of the MULTI TYPE ES WOUND HEALING FLOWCHART, at multiple uniformly spaced very short intervals, within a relatively much shorter overall timeframe compared to the much longertime point data intervals at which Rmean measurements are made and logged during each much longer overall Compare_period_n that are used to calculate the Rmean (Pbio) slope. In a preferred embodiment of this aspect of the present invention, typically, 6 Rmean values at 5 second intervals within 30 seconds are measured and logged, every 10 minutes of each stimulation cycle. The best fit linear regression slopes of these data are computed and averaged across each Compare_period_n, and thereby used as the electrode drying resistance dynamic compensation slope that is subtracted from each new Compare_period_n Rmean (Pbio) slope computation in step ML2. Importing the newest Compare_period_n Rmean values acquired and logged to the FRAM from IWP at ML1-3 of the MULTI TYPE ES WOUND HEALING FLOWCHART, using the same methodology best fit linear regression, the slope of the Rmean (Pbio) logged values for the newest Compare_period_n data points is thus computed and calibrated with compensation for electrode drying. Pbio data value variations from one measurement point to the next of greater than typically 50 kiloohms are filtered as outliers. Finally in process step ML2, the change of the new Pbio (Rmean) slope of the newest Compare_period_n compared to the previous Compare_period_n Pbio slope is computed. ML2 records and metadata are continuously logged to the FRAM memory as shown by the process line between ML2 and ML2-1.

Next is step ML3 and steps thereafter in one flow line of the algorit, and steps ML14 to the IWHP comparison graph in another flow line of the algorithm.

In step ML14, starting from when Pbio data points of the first Compare_period_n have been logged, the newest Rmean slope is compared to the Ideal Wound Healing Plot (IWHP) graph line to determine its correspondence with it. The newest data point of the last Compare_period_n data points of the current Rmean best fit linear regression line, in relation to the IWHP line, is also recorded and logged.

The IWHP graph line shows the averaged, ideal healing dynamic of Pbio when Pbio is R_wound as assigned as Rmean during the IWPs as shown in ML1-3 and when the entire function optimization algorithm is specifically applied to wound healing. Generally, the IWHP line facilitates comparison of the relative position of the current Pbio value to its known biological range. Comparison of the Rmean values and slope dynamics imported into and computed in step ML2 of the algorithm, with the slope and entire graph line of the IWHP, indicates and models both the rate and identifiable chronological stage of healing of a wound or ulcer. Time plotted as abscissa values and position are shown in arbitrary sequential time point number units, from A to DD, to allow the algorithm to self-adapt to varying rates of individual wound healing. R_wound values are plotted as ordinates in units of kiloohms based on the established medical-scientific literature of bioelectric wound healing and according to the specification disclosed in patent All 2016202751. Measured and computed Rmean values diverging from the general dynamic of the IWHP plot can thus indicate various pathological, physiological, and surgical events, including but not limited to infection, hyperemia, debridement, surgical grafts, physical (mechanical) trauma, scab and eschar formation. For example, during a wound or ulcer infection process, R_wound values decrease relatively very rapidly in comparison to the timeframes of overall wound healing stages and closure as graphed by the IWHP line and its slope.

Now describing decision process step ML3, if the new Rmean slope value imported from ML2 is a positive gradient (indicating wound healing) or if not a positive gradient is greater than the comparator value of the slope of the previous Compare_period_n data point Rmean slope (indicating wound improving from last assay), then the algorithm moves to next step ML-9 wherein the current switch set under test (SSUT) is assigned as the active switch set (ASS) if it was not already the ASS and so recorded in the ESS Score Table at step ML11. The Tested SSUT Table at ML7 is also wiped (emptied).

From step ML-9, the algorithm flow goes to next step ML-8 that is the exit point back to the MULTI TYPE ES WOUND HEALING FLOWCHART interconnection process step ML-2. In ML-8 the Rmean slope value newly computed in ML2 is assigned as the last slope value that will be used in the next future comparison in ML2 with the newest future Compare_period_n Rmean (Pbio) slope value for slope change comparison, at the beginning of the next stimulation cycle of the MULTI TYPE ES WOUND HEALING FLOWCHART when ML2 is again ‘called’. If the current SSUT has only now been assigned as the ASS in ML9 then process flow also goes to step ML10 and to the ESS Score Table ML11.

From step ML9 before exiting at ML8, in step ML13, the ASS software flag is set to its binary state of TRUE to indicate that the newest SSUT has now been assigned as the ASS and so will be output for stimulation by the MULTI TYPE ES WOUND HEALING FLOWCHART.

In step ML10, the ASS and its computed metadata are logged into the ESS Score Table ML11; if the ASS has previously been an ASS and so has an existing record already stored in Table ML11, then if its ESS start data point Rmean metadatum value is less than or equal to plus or minus SlopeMatch% of the existing record's same metadatum value, then the already logged record's metadata are updated; else if greater than plus or minus SlopeMatch% of the existing same metadatum value, an additional, separate record for the same ASS with new computed metadata is made and logged in Table ML11 and a copy record identifier code is assigned for retrieval identification purposes for when new SSUTs are tested in future process steps at ML5. In this aspect of the present invention, the SlopeMatch% comparison and other metadata computations and comparisons contribute to the algorithm being able to identify if an effective SSUT producing a beneficial Pbio vector change has a recurring relationship to a specific adverse or deterioration-causing event in the course and progress of the individual wound or ulcer healing that the algorithm is applied to.

The ESS Score Table as best shown in ML11 summarizes the metadata variables and computations of scoring for future look-up of predictive effect of previous ASSs found by the function optimization algorithm, for testing at any future point in time and stage of pathology normalization and healing of the wound, ulcer, target tissues, anatomical structure, or internal or surface lesion receiving electrotherapy Example metadata are shown. ESS_start_data_point records when the ASS first produced a therapeutic effect on Pbio (that is R_wound in the wound healing application example application of the algorithm). ESS_start_data_point_Rmean records the R_wound value when the ASS began its therapeutic effect, which can also be related by the algorithm to the IWHP graph line. Slope_before_effect_start is the value of the best fit linear regression slope (gradient) of Rmean as calculated in step ML2 for the immediately previous Compare_period_n before the ASS began its therapeutic effect ESS_slope is the absolute increase of Rmean (R_wound/Pbio) as a result of the effective ASS across the duration of the newest instance of its assignment and output by the MULTI TYPE ES WOUND HEALING FLOWCHART until the time point when it ceased being effective. ESS_statistical_score is the sum total absolute increase in Rmean (R_wound/Pbio) as a result of all instances when the ASS was assigned and output in the MULTI TYPE ES WOUND HEALING FLOWCHART in the newest Data_range duration. ML11 logged records and metadata are continuously logged to the FRAM memory as shown by the process line between ML11 and ML2-1.

Again describing decision step ML3, if neither of two conditions for new Rmean slope value obtain so that process flow cannot progress to step ML9, then the algorithm flowchart has two lines of flow, to step ML5 and onward to the next steps thereafter, and to step ML4 followed by step ML4-1.

In step ML4, the ASS software flag is assigned its binary state of FALSE to indicate that the function optimization algorithm must now select or generate a new switch set in next step ML5 in order to search for a new therapeutically effective ASS

In step ML4-1, the metatdata values of the ESS Score Table ML11 record are updated for the switch set that was last but is no longer the current ASS. The metadatam ESS_statistical_score is also computed at this step and updated in ML11.

In step ML5 the next switch set to be under test (SSUT) is selected or generated.

Selection always begins with previously successful ASSs that are recorded in the ESS Score Table ML11, which are tested sequentially in order based on their metadata matching and scoring of: match of Slope_before_start plus or minus SlopeMatch% to the newest Compare_period_n R_wound (Rmean) slope computed in ML2; if there are multiple such matches then sub-ordering is done based on ESS_statistical_score high to low, and if any such ESS_statistical_scores are 0 then these records are sub-ordered based on ESS_scores high to low. While a previous ASS retrieved and matched from the ESS Score Table ML10 is being tested, it is assigned as the current switch set under test (SSUT). Or if all recorded previous ASSs stored in ML11 have been tested unsucessfully, then a new switch set is generated from the Base Switch Set Codes Table as best shown in FIG. 6 and this new switch set is assigned as the SSUT.

Next step is ML6 wherein the SSUT is recorded and logged into the Tested SSUT Table ML7 shown with example data, in order to prevent repeat testing of a SSUT while the ASS software flag remains in the binary state of FALSE as set in process step ML4.

In next decision step ML6-1, if all possible switch sets, including both previous ASSs matched and retrieved from the ESS Score Table ML11, and new switch sets generated from the Base Switch Set Codes Table (FIG. 6), have been assigned as SSUTs and output by the MULTI TYPE ES WOUND HEALING FLOWCHART, but have all failed to beneficially affect Pbio (R_wound/Rmean) then the Default_ASS corresponding to the AcrossWound flag that is determined each time within an IWP process at ML1-3 of the MULTI TYPE ES WOUND HEALING FLOWCHART is assigned as the ASS, the Tested SSUT Table ML7 data are erased and flow goes to exit step ML-8. The Default_ASS is then output for Wound_stim_block time as timed by the DefaultASSJJmer that is started in ML6-1. Conversely, if all possible switch sets have not yet been tested, then flow only goes directly through decision step ML6-1 to exit step ML-8 without assigning the Default_ASS as the ASS and not wiping the logged ML7 data.

FIG. 6—Base Switch Set Codes Table

The first column of the Table lists the electrical stimulation (ES) components labeled corresponding to the low intensity direct current (LIDC), electric field (EF), CELLMOD (cell phenotype modification), cyclic AMP (cAMP) and cyclic GMP (cGMP) outputs of the MULTI TYPE ES WOUND HEALING FLOWCHART during its process steps, ML1-6, ML1-10, ML1-11, ML1-8 and MI1-9, respectively.

The Group 1 ES switches and Group 2 ES switches columns of the Table show the corresponding Group 1 and Group 2 binary switch states and their alphabetical codings, which are the top level and second-level switch states for the ES components that are available for switch set generation by the ML CONTROL FLOWCHART in step ML5.

In a preferred embodiment of the present invention, a default switch set named, Default_ASS, as best shown in the third Group 1 ES switches column for the example of a wound or ulcer healing application of the function optimization algorithm, is preprogrammed based on it having the highest ASS metadata scores and statistical frequency of general therapeutic effectiveness on Pbio (R_wound/Rmean) from the experience of many previous clinical multi-type ES applications involving the same target therapeutic Pbio and that has two stimulation-logical variations based on the possible placement configurations of the (+)positive and return electrodes to the wound or ulcer as specified above. The Default_ASS can be integrated into the ML CONTROL FLOWCHART in decision step ML6-1 as already described.

Group 2 ES switches, A, AB, AC, AD and AE, provide the intensities of LIDC for output in process step ML1-6.

In another aspect of the present invention, the values of Group 2 ES switch states, I_taxotype 1-4, are shown in the Taxotype-Variables box of the MULTI TYPE ES WOUND HEALING FLOWCHART, corresponding to, LIDC_bacteria, LIDC_virus, LIDC_fungus and LIDC_yeast, which have been found to have these specific microorganism taxonomic-specific bell curve characterized effect-relationships.

The medical utility of having a non binary, Group 2 ES multi-state switch, AE, has already been specified in relation to infection treatments, and in a preferred embodiment of the present invention, ranges from 1.5 to 10 microamperes in step sizes of 0.1-0.5 microamperes, for output in process step ML1-6 of the MULTI TYPE ES WOUND HEALING FLOWCHART

Group 1 switch states, G and I, correspond to second messenger stimulation process steps ML1-8 and ML19, respectively. Group 2 ES switches, GB and IB, correspond to the intra-process lines between process steps ML1-8 and ML1-9 and between process steps ML1-9 and ML1-10, of the same labeling in the MULTI TYPE ES WOUND HEALING FLOWCHART.

It will be apparent to those skilled in the art from the flowcharts, operations, computations, processes and tables shown in FIGS. 4, 5 and 6 that another defining aspect of the present invention is their integration and incorporation into the electronics and software design and functionality of the electronic stimulation device thereby providing an integrated multi-type ES treatment system controlled by the machine learning dynamic function optimization algorithm.

It should also be apparent that the integration of the function optimization algorithm with the MULTI TYPE ES WOUND HEALING FLOWCHART for the purpose of wound healing stimulation is only one of many possible such integrations with healing flowcharts for many different pathologies including but not limited to viral hepatitis, fibrosis and cirrhosis, tissue inflammation, injured tissue healing and regeneration, pain conditions especially neuropathic, treatment and normalization of a radiologically or sonographically identified internal lesion having abnormal plasia, as well as surface and internal infected tissue treatments.

A combined instance-based and regression algorithm and software code embodiment that is based on a partly true-random, dynamic function optimization task model. According to the definition that machine learning is the artificial intelligence (AI) field of the construction of algorithms and computer programs that automatically improve with their experience of performing tasks, the algorithm is a machine learning type.

Another aspect of the present invention is the application of a machine learning algorithm and executing software program to multi-type electrotherapy modality and stimulation combinations output by a single or multiple treatment devices.

In another aspect of the present invention, the environmental input data for the learning algorithm are the therapeutically desired vector quantity changes of a target bioelectric or biochemical parameter (Pbio) that is monitored and measured repeatedly electronically or otherwise every few seconds or minutes. Assessment of Pbio dynamics is performed with continuously updated regression analysis or by other statistical and analytical means. Examples of Pbio are the through-wound electrical resistance and autologous wound generated electric field of a surface wound or ulcer used as a measure of wound closure In another such example, Pbio is the complex electrical impedance (Z), capacitive reactance (Xc), inductive reactance (XL) or phase angle (F) profiles across a frequency range sweep of any tissue target that is anatomically Realizable for cross sectional planar electronic measurement invasively or non-invasively.

In another aspect of the present invention, when the function optimization algorithm is specifically applied to wound healing, Pbio can also be the electrical impedance (Z), capacitive reactance (XC), inductive reactance (XL) and phase angle (F) measured through or across the wound or ulcer.

In another aspect of the present invention multiple Pbios can be simultaneously assessed by duplicating and/or adapting sections of the function optimization algorithm.

In another aspect of the present invention, the electronic stimulation device and its software program are capable of multiple ES modalities and stimulations, five of which are termed as illustrative examples and for convenience, ES-A, ES-B, ES-C, ES-D and ES-F, although more numerous ES modality capabilities including any of those mentioned in the Background can be output by the device and software program.

In another aspect of the present invention, the function optimization task model can simultaneously optimize the functions of multiple ES modalities and stimulations.

Another aspect of the present invention is that each ES modality that the stimulation device and software are capable of has two top level binary switch states of being either switched on and output or switched off and not output by the stimulation device, which further results in the feature that when the different ES modalities are output sequentially, this series combination digital stimulation method is also capable of producing analogue combination ES patterns if one or many of the ES modalities continues to be switched off. For example, if ES-A, ES-B, ES-C and ES-D are all switched off in one stimulation combination that is repeatedly output, then modality ES-F will be output as a continuous analogue type ES.

Another aspect of the present invention is that to achieve the function optimization task the algorithm randomly at first generates and controls the electronic circuitry of the stimulation device to output one after another every possible sequential combination of ES-A, ES-B, ES-C, ES-D & ES-F, where one such example would be, ES-A_ON, ES-B_ON, ES-C_OFF, ES-D_OFF, ES-F_OFF, and where each such sequential combination thereby forms and is hereafter referred to as a switch set; and that each switch set is only output for a relatively short timeframe that is selected in relation to the biologically possible rate of variability and characteristics of Pbio, and the generation and output of all possible switch sets generated by the algorithm from all available switch state combinations is also completed within a similarly short biological timeframe also directly relative to the possible biological dynamics of Pbio.

In another aspect of the present invention the ES modalities that the stimulation device is capable of can have additional, second-level and lower level non binary switch states that allows the function optimization algorithm to generate more ES combination switch sets than the switch sets that comprise only the top level binary switch states of the ES modalities. For example, ES-A can have a range of electrical output parameter values of current, voltage or frequency depending on its ES type, where in the simplified example of ES-A being constant Output Current with three possible discrete electronic circuit intensities (1-3) then ES-A can have the additional second-level switch states of, ES-A-1, ES-A-2 and ES-A-3; whereas in reality more numerous second-level switch states for ES-A can correspond to a pre-programmed or algorithm generated gradated intensity step size through the entire range of minimum to maximum Output Current that the stimulation device can generate.

In another aspect of the present invention, the second-level switch states of constant Output Currents that correspond to gradated intensity steps through the constant Output Current range capability of the stimulation device from low intensity direct current (LIDC) up to milliampere intensity direct current (MIDC), add the capability to the function optimization algorithm to perform an automated search through the entire constant Output Current range of the stimulation device and find a second-level switch state that corresponds to a specific intensity LIDC or MIDC that has a therapeutic effect on the target localized tissue measured in terms of the positive therapeutic effect of that switch state on Pbio according to the assessment methodology of the function optimization task described above. The medical utility of this aspect of the present invention is that LIDC and MIDC can variously be applied therapeutically for the purpose of attenuating and inactivating the infection process in localized target tissue microenvironments, as for example disclosed with LIDC ES outputs in patent AU 2016202751 where a number of microorganism taxonomic-specific bell curve characterized effect-relationships were provided for viral and bacterial species. Though given the extremely large diversity within and among the different species and families of microorganisms including bacteria, protozoa, viruses, fungi and yeasts and that mutations of these microorganisms are possible and common, and because of the virtually unlimited complexities and dynamisms of tissue, cellular, and molecular microenvironments as noted above, pre programmed Output Currents that previously interacted effectively with these target tissue microenvironments to attenuate and stop the infection process involving specific microorganisms might later on in similar specific medical case instances, be ineffective. Whereas, in contrast, the solution provided by the automated search performed by the function optimization algorithm of the present invention using non binary second-level switch states of constant Output Currents can find non predictable, unknown, variable and unique-to-instance therapeutically effective Output Current intensities for theoretically any type of tissue having a microenvironmental physiological abnormality including but not limited to, bioelectric state and pathological condition involving an infection process.

In another aspect of the present invention the algorithm generated ES switch sets comprising only top level switch states and those comprising mixed level switch states are assessed dynamically in realtime for therapeutic success based on their performance effect on the continuously incoming Pbio data. The rate and limits of effect on Pbio by a switch set under test (SSUT) are computed by the algorithm as variables of the function optimization task model in order to determined if the SSUT is therapeutically successful or not. A primary score and associated metadata are computed and assigned to a successful switch set that are then recorded and logged, and the switch set is then assigned as the active switch set (ASS) to be continuously output for therapeutic stimulation. While a switch set remains the ASS the algorithm continuously updates the metadata of that ASS with experience from the stream of incoming Pbio data and its effect thereon.

In a preferred embodiment of the present invention, ASS score metadata are absolute, percentage and rate of change calculations of Pbio over pre-defined or variable time periods selected by the algorithm in relation to the involved physiological and pathological processes involving Pbio, the logged dynamics of Pbio prior to assigning the current ASS, duration of maintained therapeutic effect of the ASS on Pbio, and positional data of Pbio within its known or computed biological range of values in relation to the start and end points, and duration of the current ASS, and the specific pathology being treated.

Another aspect of the present invention is that when a successful switch set that has been assigned as the current ASS is no longer therapeutically effective at any point in time as determined by the assessment methodology already described, the algorithm first compares and matches the logged scores and metadata of previous ASSs to the recent dynamics and positional value of Pbio in order to predict their repeat successes of therapeutic effect on Pbio When a previously assigned ASS is selected in this way and then repeats its success in terms of present therapeutic effect on Pbio then it is again assigned as the current ASS, and a new record of its primary score and associated metadata are recorded; such that a single switch set can have multiple ASS assignments and corresponding records each having different primary scores and metadata. If all previous ASSs selected in this way are retested but are unsuccessful in terms of their present therapeutic effect on Pbio then the algorithm again randomly generates all possible ES switch set combinations that have not previously been assigned as ASSs and outputs and assesses each one in turn via the same methodology already described.

A resultant aspect of the present invention is that the overall function optimization task repeats until a new ASS is found.

Another key aspect of the present invention is that the function optimization algorithm that controls the combination ES switch sets output by the stimulation device, continuously learns and improves its assessment and predictive ability of what switch sets will be more or less effective at any point in time within the biological range and given the present and previous dynamics of the target Pbio from continuously incoming Pbio data and computations thereon of the current medical case it is applied to.

In another aspect of the present invention, given the individual and heterogeneous nature of many types of medical problem even when similarly classified in medical nomenclature for conventional diagnosis and identification purposes—where the reality of the uniqueness of every surface wound or ulcer is an example—advance training data for the function optimization task model of the algorithm are not applicable nor possible, and so instead and preferably the software program acquires all of its training and learning data individually and uniquely while the stimulation device is applied to each individual medical case instance.

In a variant embodiment of the present invention when there is far greater homogeneity of the medical issue and also of the related characteristics and behaviour of the selected Pbio, then training data can be used to pre-train the function optimization algorithm.

In another aspect of the present invention, in order to further overcome the obstacles to predictable and reproducible therapeutic effect with ES, the stimulation device is electronically designed to include the ability to generate ‘carrier’ base waveforms with amplitude modulation of these waveforms by secondary (‘envelope’) frequencies also termed, signal frequencies, which are often the actual active element of the this ES modality. Typical ranges of such frequencies are base frequencies in the range of 1-20,000 Hz and modulating frequencies in the range of 1-200 Hz, though these ranges are given for illustration only and are not limits to the present invention. The base frequencies are selected in order to use to advantage the complex impedance properties—particularly the bioelectrical capacitive reactance (Xc)—of the superficial tissues of the deeper localized target pathological tissues even if the more superficial tissues contain thick bones, to enable signal frequency transmission, and to match the frequency-dependent impedance profile of the internal body tissues lying between the cutaneous electrode pads in which the resultant current is induced.

In another aspect of the present invention the waveforms are generated by either direct digital synthesis (DDS) or digital to analogue (DAC) converter electronics. The advantages provided by DDS waveform generation are allowance for micro-tuning and automatic monitoring of the output with feedback adjustment; the advantages provided by DAC waveform generation are smaller physical electronic circuit area and footprint, subsequent lower manufacture cost and ease of integration with other circuits that comprise the complete electronic stimulation device.

In another aspect of the present invention the amplitude modulated waveforms generated by the electronic circuits of the stimulation device are utilized to stimulate and regulate specific intracellular second messengers, including but not limited to cyclic AMP: adenosine 3′,5′-monophosphate (cAMP), and cyclic GMP: guanosine 3′,5′-cyclic monophosphate (cGMP) The chemical pathways, interactions and number of functional processes regulated by these second messengers are very large and extensively studied in the medical-scientific literature and so are not enumerated here. The medical advantage of this utilization is that in many medical instances its inclusion eliminates much of the uncertainty about the appropriateness and predicted effectiveness of the ES, since the functional processes regulated by the second messengers are already known in great detail and activation by them therefore far more predictable. A further medical advantage is that with targeted stimulation of specific second messengers, the ES is essentially, directly boosting what the body is already doing to normalize virtually any pathology at hand, without the human medical physician or technician needing to make extremely complex decisions for the best ES treatment strategy across different timeframes and in relation to the specific nature of the pathology, and further, without risking the disruption nor blocking of normal physiologic healing processes.

In a preferred embodiment of the present invention, the stimulation device can operate as a constant voltage source outputting the DDS or DAC generated amplitude modulated waveforms that results in clinically effective ES even through thick bone at very low Output Voltages typically at the lower end of the 1-200 millivolt range and often as low as 70 millivolts, which is far below the levels of Output Voltages of waveform generating technologies and devices in the prior art necessary for them to give comparative therapeutic effects under the same conditions.

Another aspect of the present invention in relation to intracellular second messenger stimulation is that the software of the stimulation device generates a repeating, timed stimulation cycle that includes an up-regulating component that increases the production of a specific second messenger, followed by the possibility of a first rest period of variable programmable duration, then followed by a component that stimulates the pathway activation of that second messenger, and then again by the option of a second rest period also of programmable variable duration, when the whole second messenger stimulation cycle then repeats from the beginning. The present invention discloses the following base and signal frequencies confirmed during the clinical and laboratory research of the inventors: 4000 Hz modulated by 10 Hz for up-regulating production of cAMP (labeled cAMP_HZ1 in process step ML1-8 of the MULTI TYPE ES WOUND HEALING FLOWCHART shown in FIG. 5), followed by 4000 Hz modulated by 20 Hz for increasing pathway activation and utilization of cAMP (labeled cAMP_HZ2 in process step ML1-8 shown in FIG. 5), and, 4000 Hz modulated by 25 Hz for up-regulating cGMP production (labeled cGMP_HZ1 in process step ML1-9 of FIG. 5) followed by 4000 Hz modulated by 20 Hz for increasing pathway activation and utilization of cGMP (labeled cGMP_HZ2 in process step ML1-9 of FIG. 5). Though it should be understood by those skilled in the art that this aspect of the present invention is not limited to stimulation and regulation of only cAMP and cGMP and that the basic ES principle herewith disclosed can be applied to the stimulation of any number of other second messengers. Furthermore, the basic logic and biological effectiveness of stimulation of second messengers and especially of cAMP by means of chemical (drug) intervention is well and generally established, whereas the present invention discloses a dedicated electromedical approach that can achieve these same results.

In a preferred embodiment of the present invention, the first rest period following the production component and the second rest period following the pathway activation stimulation component of the stimulation cycle are typically of 1-3 minutes duration in order to maximize the overall biostimulation effect by allowing the electrochemical intracellular second messenger processes to physiologically synchronize with the stimulation cycles, and not be overwhelmed by the electrical energy that is a common pitfall of many ES approaches.

In another preferred embodiment of the present invention, the ES combination switch sets already described include all possible combinations of specific intracellular second messenger stimulations, comprising top level binary switch states of on and off, and binary second-level switch states for production upregulation stimulation followed or not by rest period and pathway activation stimulation. The medical advantage of these second-level switch states is the ability to regulate pathway activation of second messengers such as cAMP that have mediating and controlling effects on various bio electrochemical dependencies such as keratinocyte directional migration under the influence of an electric field as autologously generated by a healing wound having sufficient normal transepithelial electrical potential.

To illustrate the general medical and electrotherapeutic advantages of the intracellular second messenger ES feature of the present invention, various well established beneficial therapeutic effects of cAMP production and pathway activation up-regulation are summarized, as examples only that should be understood do not describe limits to the scope of this aspect of the present invention: Decrease of the overall activity and the specific electrical voltage across nociceptor membrane ion channels, reducing or stopping the transmission (blockade) of mostly neuropathic pain signals, rather than overwhelming the natural pain generating process as with transcutaneous electrical nerve stimulation devices;

Blocking small diameter, mostly C type fiber motor and sensory nerves, resulting in vasodilation of arterioles, increased circulation, less perceived pain, local muscle relaxation, increased flushing of toxic metabolites, increased nutrient, enzyme and hormone uptake, and less neurogenic inflammation;

Up-regulation of metabolic activity for injury healing including gene expression;

Increase in neuron and axon survival and regeneration following injury;

Immune and non immune system inflammation modulating effects giving immediate and long-term protection of injured tissues;

Reduction of muscle tension and central nervous system modulated anti-spastic action following injury;

Increased functional stimulation and regeneration of muscle, and peripheral, and possibly, central, nerve tissue;

Enhancement of whole brain and synapse plasticity following injury;

Apoptosis stimulation;

Supporting medical-scientific literature

The invention can include claims

1. A multi-type electrotherapy system for medical treatment of one or more of: surface and internal infected tissues, surface wounds and ulcers, trauma injuries, abnormal plasia and fibrotic changes, and pain conditions, the system comprising: a machine learning algorithm, and an electrical stimulation (ES) device electronically designed and software programmed with the learning algorithm and to include the ability to generate carrier base waveforms with amplitude modulation of the carrier base waveforms by secondary frequencies.

2. The system of claim 1 wherein the machine learning algorithm is a dynamic function optimization task algorithm.

3. The system of claim 1 wherein input data for the learning algorithm includes therapeutically desired vector quantity changes of a target bioelectric or biochemical parameter (Pbio) that is monitored and measured repeatedly electronically or chemically every few seconds or minutes.

4. The system of claim 3 wherein assessment of Pbio change is performed with continuously updated regression analysis or by other statistical and analytical means

5. The system of claim 1 wherein the ranges of generated carrier base waveform frequencies by the ES device are base frequencies in the range of 1-20,000 Hz and the secondary frequencies are amplitude modulating signal frequencies in the range of 1-200 Hz.

6. The system of claim 5 wherein the waveforms are generated by either direct digital synthesis (DDS) or digital to analogue (DAC) converter electronics.

7. The system of claim 1 wherein the amplitude modulated waveforms generated by the electronic circuits of the stimulation device are utilized to stimulate and regulate specific intracellular second messengers.

8. The system of claim 7 wherein the second messengers include cyclic AMP: adenosine 3′,5′-monophosphate (cAMP), and cyclic GMP: guanosine 3′,5′-cyclic monophosphate (cGMP).

9. The system of claim 6 wherein the stimulation device can operate as a constant voltage source outputting the DDS or DAC generated amplitude modulated waveforms that results in clinically effective ES.

10. The system of claim 1 wherein ES is performed through dense tissues and bone at very low Output Voltages.

11. The system of claim 10 wherein the OutputVoltages are at the lower end of the millivolt range.

12. The system of claim 7 wherein for intracellular second messenger stimulation, the software of the stimulation device generates a repeating, timed stimulation cycle that includes an up-regulating component that increases the production of a specific second messenger.

13. The system of claim 12 wherein the step is followed by a first rest period of variable programmable duration, then followed by a component that stimulates the activation of the second messenger.

14. The system of claim 13 wherein the step is followed by a second rest period also of programmable variable duration, after which the complete second messenger stimulation cycle then repeats from the beginning.

15. The system of claim 12 wherein the base and modulating signal frequencies comprise: a base frequency that matches the frequency-dependent impedance profile of the internal body tissues through which the resultant current is induced amplitude modulated by 10 Hz for up-regulating production, followed by the same base frequency amplitude modulated by 20 Hz for increasing activation and utilization of cAMP, and, modulated by 25 Hz for up-regulating production, followed by amplitude modulation by 20 Hz for increasing activation and utilization of cGMP.

16. The system of claim 14 wherein the first rest period following the production component and the second rest period following the activation component of the stimulation cycle are typically of 1-3 minutes duration in order to enable the overall biostimulation effect by allowing the intracellular second messenger processes to physiologically synchronize with the stimulation components and cycles.

17. The system of claim 3 wherein all possible multi-type ES combinations are derived from a switch set table of base data comprising top level binary switch states of stimulation on and off, and non binary second-level switch states consisting of the full ranges of each of the different ES types.

18. The system of claim 17 wherein the function optimization task algorithm tests, scores and selects ES switch sets based on their relative effects on Pbio.

19. The system of claim 17 wherein the learning algorithm continuously analyzes, updates, logs and accesses the tested switch set scoring history to improve its switch set selection for performance of task optimization.

20. The system of claim 19 wherein the ferroelectric RAM memory of multiple ES devices are a continuously accumulating, shared knowledge-base, uploaded wirelessly and automatically from each ES device to a central or distributed electronic database that each new and in service device accesses and utilizes in its learning algorithm.

The invention also includes other claims.

1. A multi-type combination electrical stimulation method of medical treatment of any one of: surface and internal infected tissues, surface wounds and ulcers, trauma injuries, abnormal plasia and fibrotic changes, and pain conditions, the method comprising:

providing a vector bioparameter monitoring, measuring and electrical stimulation device, connected to measurement and stimulation parameter integrated material construction electrode pads, capable of multi-type combination electrical stimulation,

placing the electrode pads across or anatomically cross-sectioning or bioelectrically topographically matching a target location being a target physical lesion, tissue or anatomical structure,

measuring a bioelectric or biochemical vector bioparameter of the target location using the device and electrode pads at repeating intervals to generate measurement data, analytically calculating vector bioparameter dynamics data from the measurement data, and

concurrently repeatedly measuring and analytically calculating compensation data that represent the electrochemical, electromechanical, environmental and body temperature, humidity, and skin condition changes affecting the measurement and stimulation electrode pad interfaces with the body using the device and electrode pads, applying the compensation data to the vector bioparameter dynamics data to generate compensated vector bioparameter dynamics data that represent the actual physiological change of the vector bioparameter separated from the compensation data, and

determining a stimulation time period that relates to the vector bioparameter at the target location, the selection, timing and sequencing of the multi-type combination and parameter electrical stimulation of the target location during the next stimulation time period then being determined by the compensated vector bioparameter dynamics data of the previous stimulation time period and statistically and analytically across multiple previous stimulation time periods, resulting from the effects of the previous multi-type electrical stimulation combinations and parameters applied to the target location, and compared to a biologically and theoretical modeled improving dynamic of the vector bioparameter of the specific lesion or pathology at the target location.

2. The method of claim 1 wherein the multi-type electrical stimulation combinations generated by the device include low intensity direct constant current, constant millivolt voltage peak amplitude modulated waveform, and constant millivolt static electric field with reversible polarity, all with variable ranges and parameters.

3. The method of claim 2 wherein the waveform type of electrical stimulation includes the ability to generate sequenced and timed carrier base frequency waveforms with amplitude modulation of the carrier base waveforms by secondary frequencies.

4. The method of claim 1 wherein the durations of the measurement periods and intervals of the vector bioparameter measurement data and of the compensation data, and the duration of the stimulation time period, are separately adjusted to the instrument detectable limits and measurement resolution, and actual rates of changes, to enable the separated measurements and acquisition of the data sets.

5. The method of claim 1 wherein the repeated selection of effective multi-type electrical stimulation combinations and parameters continuously improves based on previous compensated bioparameter dynamics data behavior under stimulation, with changes in the target location over time and its physiological and pathological course and events.

6. The method of claim 5 wherein assessment of the compensated vector bioparameter dynamics data and the compensation data changes are performed with continuously updated regression analysis or by other statistical and analytical means.

7. The method of claim 2 wherein all possible multi-type electrical stimulation and parameter combinations are derived from a switch set table comprising top level binary switch states of stimulation type on or off, and non binary second level switch states consisting of the full ranges of parameters of each of the different electrical stimulation types.

8. The method of claim 3 wherein during waveform type electrical stimulation the device generated carrier base waveform frequencies are in the range of 1-20,000 Hz and the amplitude modulating signal frequencies are in the range of 1-200 Hz.

9. The method of claim 3 wherein the amplitude modulated waveforms, sequencing and timings generated by the stimulation device are utilized as specific bioinformational codes that select and target the regulation of individual intracellular second messengers.

10. The method of claim 8 wherein the carrier base waveform frequency is calculated based on the previously measured overall frequency dependent complex impedance profile of the internal body tissues between the electrode pads, and wherein the voltage peak amplitude of the carrier base waveform is repeatedly and automatically analytically calculated and varied by the device based on the complex impedance profile to maintain a constant resultant alternating current through the body and the target location.

11. The method of claim 9 wherein the second messengers include cyclic AMP: adenosine 3′,5′-monophosphate, and cyclic GMP: guanosine 3′,5′-cyclic monophosphate.

12. The method of claim 9 wherein the stimulation device generates a repeating, timed stimulation sequence and cycle that includes a signal frequency amplitude modulated carrier base frequency waveform that signals the up-regulation of production of the specific second messenger, followed by a first rest period of variable duration.

13. The method of claim 12 wherein the step is followed by the carrier base frequency waveform differently signal frequency amplitude modulated that signals the activation and utilization of the specific second messenger, then followed by a second rest period also of variable duration.

14. The method of claim 9 wherein the carrier base frequency waveform is amplitude modulated by a signal frequency of 10 Hz for up-regulating production, followed by the same carrier base frequency waveform amplitude modulated by a signal frequency of 20 Hz for increasing utilization of cyclic AMP; and where the carrier base frequency waveform is signal frequency modulated by 25 Hz for up-regulating production, followed by the same carrier base frequency waveform signal frequency amplitude modulated by 20 Hz for increasing utilization of cyclic GMP.

15. The method of claims 12 and 13 wherein the rest periods are typically of 1-3 minutes duration that empirically critically enable and optimize the overall intracellular second messenger biostimulation.

Silver Iontophoresis:
Becker R O, Spadaro J A. Treatment of orthopaedic infections with electrically generated silver ions. A preliminary report. J Bone Joint Surg Am. 1978 October; 60(7):871-81.
Nand S, Sengar G K, Nand S, Jain V K, Gupta T D. Dual use of silver for management of chronic bone infections and infected non-unions. J Indian Med Assoc. 1996 March; 94(3):91-5.
Webster D A, Spadaro J A, Becker R O, Kramer S. Silver anode treatment of chronic osteomyelitis. Clin Orthop Relat Res. 1981 November-December; (161):105-14.
Becker R O. Processes and products involving cell modification. U.S. Pat. No. 4,528,265 A. Jul. 9, 1985.
Becker R O, Flick A B, Becker A J. Iontopheretic system for stimulation of tissue healing and regeneration. U.S. Pat. No. 5,814,094 A. Sep. 29, 1998.
Chu C S, McManus A T, Pruitt B A Jr, Mason A D Jr. Therapeutic effects of silver nylon dressings with weak direct current on Pseudomonas aeruginosa-infected burn wounds. J Trauma 1988 October; 28(10):1488-92.
Satyanand, Saxena A K, Agarwal A. Silver iontophoresis in chronic osteomyelitis. J Indian Med Assoc. 1986 May; 84(5): 134-6.
Uezono H. Effect of weak direct current with silver electrodes on bacterial growth. Nihon Seikeigeka Gakkai Zasshi. 1990 September; 64(9):860-7. Department of Orthopaedic Surgery, Faculty of Medicine, Kagoshima University, Japan
Raad I, Flachem R, Zermeno A, Stephens L C, Bodey G P. Silver iontophoretic catheter: a prototype of a long-term antiinfective vascular access device. J Infect Dis. 1996 February; 173(2):495-8.
Schwass D R, Meledandri C J. Enhanced Penetration of Silver Nanocomposite Assemblies into Dentine Using Iontophoresis: Toward the Treatment of Dental Caries. ChemPlusChem 2014, 79, 1671-1675.

Low Voltage Iontophoresis:
Chizmadzhev Y A, Indenbom A V, Kuzmin P I, Galichenko S V, Weaver J C, Potts R O. Electrical properties of skin at moderate voltages: contribution of appendageal macropores. Biophys J. 1998 February; 74(2 Pt 1):843-56.
Kasting G B, Bowman L A. D C electrical properties of frozen, excised human skin. Pharm Res. 1990 February; 7(2): 134-43.

Silver-Nylon Effective Antimicrobial Device:
Deitch E A, Marino A A, Malakanok V, Albright J A. Silver nylon cloth: in vitro and in vivo evaluation of antimicrobial activity. J Trauma. 1987 March; 27(3):301-4.
Becker R O. Silver ions in the treatment of local infections. Met Based Drugs. 1999; 6(4-5):311-4.
MacKeen P C, Person S, Warner S C, Snipes W, Stevens JrSE. Silver-coated nylon fiber as an antibacterial agent. Antimicrob Agents Chemother. January 1987; 31(1): 93-99.
Deitch E A, Marino A A, Gillespie T E, Albright J A. Silver-nylon: a new antimicrobial agent. Antimicrob Agents Chemother. 1983 March; 23(3):356-9.
Krieger B R, Davis D M, Sanchez J E, Mateka J J, Nfonsam V N, Frattini J C, Marcet J E. The use of silver nylon in preventing surgical site infections following colon and rectal surgery. Dis Colon Rectum. 2011 August; 54(8):1014-9.
Barillo D J, Pozza M, Margaret-Brandt M. A literature review of the military uses of silver-nylon dressings with emphasis on wartime operations. Burns. 2014 December; 40 Suppl 1:S24-9.
Abboud E C, Settle J C, Legare T B, Marcet J E, Barillo D3, Sanchez J E. Silver-based dressings for the reduction of surgical site infection: review of current experience and recommendation for future studies. Burns. 2014 December; 40 Suppl 1:S30-9.
Becker R O Silver ions in the treatment of local infections Met Based Drugs 1999; 6(4-5):311-4.

Silver Ions and Nanoparticles Effects on Bacteria and Viruses:
Spadaro J A, Berger T J, Barranco S D, Chapin S E, Becker R O. Antibacterial Effects of Silver Electrodes with Weak Direct Current. Antimicrobial Agents and Chemotherapy 1974; 6(5):637-642.
Berger T J, Spadaro J A, Chapin S E, Becker R O Electrically Generated Silver Ions: Quantitative Effects on Bacterial and Mammalian Cells. Antimicrobial Agents and Chemotherapy 1976; 9(2):357-358.
Morones-Ramirez J R, Winkler J A, Spina C S, Collins J J. Silver Enhances Antibiotic Activity Against Gram-negative Bacteria. Science translational medicine 2013; 5(190): 190ra81.
Jung W K, Koo H C, Kim K W, Shin S, Kim S H, Park Y H. Antibacterial activity and mechanism of action of the silver ion in Staphylococcus aureus and Escherichia coli. Appl Environ Microbiol. 2008 April; 74(7):2171-8.
Liau S Y, Read D C, Pugh W J, Furr J R, Russell A D. Interaction of silver nitrate with readily identifiable groups: relationship to the antibacterial action of silver ions. Lett Appl Microbiol. 1997 October; 25(4):279-83.
Xiu Z M, Zhang Q B, Puppala H L, Colvin V L, Alvarez P J. Negligible particle-specific antibacterial activity of silver nanoparticles. Nano Lett. 2012 Aug. 8; 12(8):4271-5. Epub 2012 Jul. 9.
Rai M, Kon K, Ingle A, Duran N, Galdiero S, Galdiero M Broad-spectrum bioactivities of silver nanoparticles: the emerging trends and future prospects. Appl Microbiol Biotechnol. 2014 March; 98(5):1951-61. Epub 2014 Jan. 10.
Chen N, Zheng Y, Yin J, Li X, Zheng C. Inhibitory effects of silver nanoparticles against adenovirus type 3 in vitro. J Virol Methods. 2013 November; 193(2):470-7. Epub 2013 Jul. 22.
Lara H H, Ayala-Nuhez N V, Ixtepan-Turrent L, Rodriguez-Padilla C. Mode of antiviral action of silver nanoparticles against HIV-1. Journal of Nanobiotechnology 2010; 8: 1.
Gaikwad S, Ingle A, Gade A, Rai M, Falanga A, Incoronato N, Russo L, Galdiero S, Galdiero M. Antiviral activity of mycosynthesized silver nanoparticles against herpes simplex virus and human parainfluenza virus type 3. International Journal of Nanomedicine 2013; 8:4303-4314.
Elechiguerra J L, Burt J L, Morones J R, Camacho-Bragado A, Gao X, Lara H H, Yacaman M J. Interaction of silver nanoparticles with HIV-1. J Nanobiotechnology. 2005 Jun. 29; 3:6.
Lansdown A B. Silver in health care: antimicrobial effects and safety in use. Curr Probl Dermatol. 2006; 33:17-34.
Silver S. Bacterial silver resistance: molecular biology and uses and misuses of silver compounds. FEMS Microbiol Rev. 2003 June; 27(2-3):341-53.

DC Antimicrobial Effects Including Viral Deactivation and Infection Inhibition:
Sandvik E L, McLeod B R, Parker A E, Stewart P S. Direct electric current treatment under physiologic saline conditions kills Staphylococcus epidermidis biofilms via electrolytic generation of hypochlorous acid. PLoS One. 2013; 8(2):e55118. doi: 10.1371/journal. pone.0055118. Epub 2013 Feb. 4.
Ruiz-Ruigomez M, Badiola J, Schmidt-Malan S M, Greenwood-Quaintance K, Karau M J, Brinkman C L, Mandrekar J N, Patel R. Direct electrical current reduces bacterial and yeast biofilm formation. International Journal of Bacteriology. Volume 2016 (2016).
Liu W K, Brown M R, Elliott T S. Mechanisms of the bactericidal activity of low amperage electric current (D C). J Antimicrob Chemother. 1997 June; 39(6):687-95.
Brinkman C L, Schmidt-Malan S M, Karau M J, Greenwood-Quaintance K, Hassett D J, Mandrekar J N, Patel R. Exposure of Bacterial Biofilms to Electrical Current Leads to Cell Death Mediated in Part by Reactive Oxygen Species. PLoS One 2016 Dec. 19; 11(12):e0168595. doi: 10.1371/journal. pone 0168595. eCollection 2016.
Schmidt-Malan S M, Karau M J, Cede J, Greenwood-Quaintance K E, Brinkman C L, Mandrekar J N, Patel R. Antibiofilm activity of low-amperage continuous and intermittent direct electrical current. Antimicrob Agents Chemother. 2015 August; 59(8):4610-5.
Schmidt-Malan S M, Brinkman C L, Greenwood-Quaintance K E, Karau M J, Mandrekar J N, Patel R. Activity of Electrical Current in Experimental Propionibacterium acnes Foreign-Body Osteomyelitis. Antimicrobial Agents and Chemotherapy. 2017; 61(2):e01863-16.
Del Pozo J L, Rouse M S, Euba G, Kang C I, Mandrekar J N, Steckelberg J M, Patel R. The electricidal effect is active in an experimental model of Staphylococcus epidermidis chronic foreign body osteomyelitis. Antimicrob Agents Chemother. 2009 October; 53(10):4064-8. doi: 10.1128/AAC.00432-09. Epub 2009 Aug. 3.
Rowley B A, McKenna J M, Chase G R, Wolcott L E. The influence of electrical currents on infecting microorganism in wounds. Ann N Y Acad Sci. 1974; 238:543-51.
Kalinowski D P, Edsberg L E, Flewson R A, Johnson R H, Brogan M S. Low-voltage direct current as a fungicidal agent for treating onychomycosis. J Am Podiatr Med Assoc. 2004 November-December; 94(6):565-72.
Thibodeau E A, Handelman S L, Marquis R E. Inhibition and killing of oral bacteria by silver ions generated with low intensity direct current. J Dent Res. 1978 September-October; 57(9-10):922-6.
Flail R E, Bender G, Marquis R E. In vitro effects of low intensity direct current generated silver on eukaryotic cells J Oral Maxillofac Surg. 1988 February; 46(2):128-33.
Tronstad L, Trope M, Flammond B F. Effect of electric current and silver electrodes on oral bacteria. Endod Dent Traumatol. 1985 June; 1(3):112-5.
Floare J I, Rajnicek A M, McCaig C D, Barker R N, Wilson F I M. Electric fields are novel determinants of human macrophage functions J Leukoc Biol. 2016 June; 99(6):1141-51. doi: 10.1189/jlb.3A0815-390R.
Merriman H L, Hegyi C A, Albright-Overton C R, Carlos J Jr, Putnam R W, Mulcare J A. A comparison of four electrical stimulation types on Staphylococcus aureus growth in vitro. J Rehabil Res Dev. 2004; 41 (2): 139-146.
Schmidt-Malan S M, Brinkman C L, Karau M J, Brown R A, Waletzki B E, Berglund L J, Schuetz A N, Greenwood-Quaintance K E, Mandrekar J N, Patel R. Effect of Direct Electrical Current on Bones Infected with Staphylococcus epidermidis. JBMR Plus, 3: e10119. doi:10.1002/jbm4.10119.
Kim Y W, Subramanian S, Gerasopoulos K, Ben-Yoav H, Wu H C, Quan D, Carter K, Meyer M T, Bentley W E, Ghodssi R. Effect of electrical energy on the efficacy of biofilm treatment using the bioelectric effect. NPJ Biofilms Microbiomes. 2015 Sep. 23; 1: 15016.
Roohandeh M, Bamdad T. Inactivation of herpes simplex virus type 1 & adenovirus type 5 by direct electric current at a biocompatible level in vitro. Clin Lab. 2011; 57(7-8):489-95.
Kumagai E, Tominaga M, Harada S. Sensitivity to electrical stimulation of human immunodeficiency virus type 1 and MAGIC-5 cells. AMB Express. 2011; 1 (1):23. Published 2011 Aug. 8. doi: 10.1186/2191-0855-1-23.

Apoptosis Effects:
Warten berg M, Wirtz N, Grab A, Niedermeier W, Hescheler J, Peters S C, Sauer H. Direct current electrical fields induce apoptosis in oral mucosa cancer cells by NADPH oxidase-derived reactive oxygen species. Bioelectromagnetics. 2008 January; 29(1):47-54.
Kurokawa M, Sakagami H, Kokubu F, Noda H, Takeda M, Adachi M. Induction of apoptotic cell death by direct-current treatment in human leukemic cell lines. J Cancer Res Clin Oncol. 1997; 123(7):370-6.
Kim H B, Ahn S, Sim S B Apoptosis by direct electric field (DEF) and nanosecond pulsed electric field (nsPEF) in tumor cells and tumor tissues. The 30th International Conference on Plasma Science, 2003. ICOPS 2003. IEEE Conference Record—Abstracts, Jeju, South Korea, 2003, pp. 436-(299) Nakamura Y, Takahashi K, Shimetani A, Sakagami H, Nishikawa H. Cytotoxicity of Direct Current with Antibacterial Agents against Host Cells In Vitro. J Endod. 2005 October; 31 (10):755-8.
Veiga V F, Nimrichter L, Teixeira C A, Morales M M, Alviano C S, Rodrigues M L, Holandino C. Exposure of human leukemic cells to direct electric current: generation of toxic compounds inducing cell death by different mechanisms. Cell Biochem Biophys. 2005; 42(1):61-74.
David S L, Absolom D R, Smith C R, Gams J, Herbert M A. Effect of low level direct current on in vivo tumor growth in hamsters. Cancer Res. 1985 November; 45(11 Pt 2):5625-31.
Turler A, Schaefer H, Schaefer N, Wagner M, Maintz D, Qiao J C, Hoelscher A H. Experimental low-level direct current therapy in liver metastases: influence of polarity and current dose. Bioelectromagnetics. 2000 July; 21(5):395-401.
Jarm T, Cemazar M, Steinberg F, Streffer C, Sersa G, Miklavcic D. Perturbation of blood flow as a mechanism of anti-tumour action of direct current electrotherapy. Physiol Meas. 2003 February; 24(1):75-90.
Schaefer N, Schafer H, Maintz D, Wagner M, Overhaus M, Hoelscher A H, TQrler A. Efficacy of direct electrical current therapy and laser-induced interstitial thermotherapy in local treatment of hepatic colorectal metastases: an experimental model in the rat. J Surg Res. 2008 May 15; 146(2):230-40. Epub 2007 Aug. 8.
Griffin D T, Dodd N J, Moore J V, Pullan B R, Taylor T V. The effects of low-level direct current therapy on a preclinical mammary carcinoma: tumour regression and systemic biochemical sequelae. British Journal of Cancer. 1994; 69(5):875-878.
Miklavcic″ D, Jarm T, C″emaz″ar M, Sers″a G, An D J, Belehradek J, Mir L M. Tumor treatment by direct electric current Tumor perfusion changes. Bioelectrochemistry and Bioenergetics. Volume 43, Issue 2, August 1997, Pages 253-256.
Miklavcic D, Semrov D, Valencic V, Sersa G, Vodovnik L. Tumor Treatment by Direct Electric Current: Computation of Electric Current and Power Density Distribution. Electro- and Magnetobiology, Volume 16, 1997—Issue 2.
Jimenez R P, Pupo A E, Cabrales J M, Joa J A, Cabrales L E, Nava J J, Aguilera A R, Mateus M A, Jarque M V, Brooks S C 3D Stationary electric current density in a spherical tumor treated with low direct current: an analytical solution. Bioelectromagnetics. 2011 February; 32(2): 120-30.
Ciria H M C, Gonzalez M M, Zamora L O, et al. Antitumor effects of electrochemical treatment Chin J Cancer Res. 2013 April; 25(2): 223-234.

DC Microcurrent Therapy:
Gokal R, Armstrong K, Durant J, Todorsky W, Miller L. The Successful Treatment of Chronic Pain Using Microcurrent Point Stimulation Applied to Scars. Int J Comp Alt Med 10(3): 00333, 2017.
Chevalier A, Armstrong K, Gokal R. Detailed Heart Rate Variability, Exercise Tolerance, Cortical and Vas Pain Scale Analysis of Two Forms of Electro-Therapy Applied To A Patient with Chronic Back Neuropathic Pain. J Cell Mol Biol 2017 1:001.
Lee B Y, A L-Waili N, Stubbs D, Wendell K, Butler G, A L-Waili T, A L-Waili A. Ultra-low microcurrent in the management of diabetes mellitus, hypertension and chronic wounds: Report of twelve cases and discussion of mechanism of action. Int J Medical Sciences. 2010; 7(1):29-35.
Fujiya H, Ogura Y, Ohno Y, Goto A, Nakamura A, Ohashi K, Uematsu D, Aoki H, Musha H, Goto K. Microcurrent electrical neuromuscular stimulation facilitates regeneration of injured skeletal muscle in mice. J Sports Sci Med. 2015 May 8; 14(2):297-303.
Fujiya H, Goto K. New aspects of microcurrent electrical neuromuscular stimulation in sports medicine. The Journal of Physical Fitness and Sports Medicine. 2016 Volume 5 Issue 1 Pages 69-72.
Electrotherapy: evidence-based practice, edited by Tim Watson. Churchhill Livingstone, Elsevier Ltd, 2008, pages 41-45.
Becker R O. Evidence for a primitive D C electrical analog system controlling brain function. Subtle Energies 2, 71-88. Subtle Energies 2, 71-88, 1991.

Dielectrophoresis:
ArcherS, Rixon F J, Morgan H Electrorotation studies of baby hamster kidney fibroblasts infected with herpes simplex virus type 1. Biophys J. 1999 May; 76(5): 2833-2842.
Bonincontro A, Melucci-Vigo G, Risuleo G. Biosci Rep. Mouse polyomavirus mediated effects on the infected cell membrane studied by dielectric spectroscopy. J Microw Power Electromagn Energy. 2001; 36(2):67-75.
Berardi V, Aiello C, Bonincontro A, Risuleo G. Alterations of the plasma membrane caused by murine polyomavirus proliferation: an electrorotation study. J Membr Biol. 2009; 229(1):19-25. doi: 10.1007/s00232-009-9172-6.

Effects on Antibiotic Resistant Bacteria:
Morones-Ramirez J R, Winkler J A, Spina C S, Collins J J. Silver Enhances Antibiotic Activity Against Gram-negative Bacteria Science translational medicine. 2013; 5(190): 190ra81.
Rai M, Kon K, Ingle A, Duran N, Galdiero S, Galdiero M. Broad-spectrum bioactivities of silver nanoparticles: the emerging trends and future prospects. Appl Microbiol Biotechnol. 2014 March; 98(5):1951-61. Epub 2014 Jan. 10.
Lara H H, Ayala-NCihez N V, Turrent L-D-C I, Padilla C R. Bactericidal effect of silver nanoparticles against multidrug-resistant bacteria World Journal of Microbiology and Biotechnology. April 2010, Volume 26, Issue 4, pp 615-621.
Rai M K, Deshmukh S D, Ingle A P, Gade A K. Silver nanoparticles: the powerful nanoweapon against multidrug-resistant bacteria. Journal of Applied Microbiology, 112: 841-852, 2012.
Chu C S, McManus A T, Pruitt B A Jr, Mason A D Jr, Jung W K, Koo H C, Kim K W, Shin S, Kim S H, Park Y H. Antibacterial activity and mechanism of action of the silver ion in Staphylococcus aureus and Escherichia coli. Appl Environ Microbiol 2008 April; 74(7):2171-8.
Thapa R, Bhagat C, Shrestha P, Awal S, Dudhagara P. Enzyme-mediated formulation of stable elliptical silver nanoparticles tested against clinical pathogens and MDR bacteria and development of antimicrobial surgical thread. Annals of Clinical Microbiology and Antimicrobials. 2017; 16:39.
Lkhagvajav N, Yasa I. Celik E, Koizhaiganova M, Sari O. Antimicrobial activity of colloidal silver nanoparticles prepared by sol-gel method. Digest Journal of Nanomaterials and Biostructures Vol. 6, No 1, January-March 2011, p. 149-154.
Gupta L K, Jindal R, Beri H K, Chhibber S. Virulence of silver-resistant mutant of Klebsiella pneumoniae in bum wound model. Folia Microbiol (Praha). 1992; 37(4):245-8.
Kalan L R, Pepin D M, Ul-Haq I, Miller S B, Hay M E, Precht R J. Targeting biofilms of multidrug-resistant bacteria with silver oxynitrate. Int J Antimicrob Agents. 2017 June; 49(6):719-726.
Panacek A, Smekalova M, Vecefova R, Bogdanova K, Roderova M, Kolaf M, Kilianova M, Hradilova S, Froning J P, Havrdova M, Prucek R, Zbofil R, Kvitek L. Silver nanoparticles strongly enhance and restore bactericidal activity of inactive antibiotics against multiresistant Enterobacteriaceae. Colloids Surf B Biointerfaces. 2016 Jun. 1; 142:392-9.
Cavassin E D, de Figueiredo L F, Otoch J P, Seckler M M, de Oliveira R A, Franco F F, Marangoni V S, Zucolotto V, Levin A S, Costa S F. Comparison of methods to detect the in vitro activity of silver nanoparticles (AgNP) against multidrug resistant bacteria. J Nanobiotechnology. 2015 Oct. 5; 13:64.
Yuan Y G, Peng Q L, Gurunathan S. Effects of Silver Nanoparticles on Multiple Drug-Resistant Strains of Staphylococcus aureus and Pseudomonas aeruginosa from Mastitis-Infected Goats: An Alternative Approach for Antimicrobial Therapy. Int J Mol Sci. 2017 Mar. 6; 18(3).
Zou L, Lu J, Wang J, Ren X, Zhang L, Gao Y, Rottenberg M E, Flolmgren A. Synergistic antibacterial effect of silver and ebselen against multidrug-resistant Gram-negative bacterial infections. EMBO Mol Med. 2017 Jun. 12.
Percival S L, Thomas J, Linton S, Okel T, Corum L, Slone W. The antimicrobial efficacy of silver on antibiotic-resistant bacteria isolated from burn wounds. Int Wound J. 2012 October; 9(5):488-93.

Bioelectric Wound Healing:
Illingworth C M, Barker A T. Measurement of electrical currents emerging during the regeneration of amputated fingertips in children. Clin. Phys. Physiol. Meas. 1 87, 1980.
Barker A T, Jaffe L F, Vanable J W Jr The glabrous epidermis of cavies contains a powerful battery. Am J Physiol 1982 March; 242(3):R358-66.
LerCinovic A, Bobanovic F, Vodovnik L. Endogenous potentials in two different models of human skin injuries. Bioelectrochemistry and Bioenergetics, 30 (1993) 221-227.
Nishimura K Y, Isseroff R R, Nuccitelli R. Human keratinocytes migrate to the negative pole in direct current electric fields comparable to those measured in mammalian wounds. J Cell Sci. 1996 January; 109 (Pt 1): 199-207.
Spence D W, Pomeranz B. Surgical wound healing monitored repeatedly in vivo using electrical resistance of the epidermis. Physiol Meas. 1996 May; 17(2):57-69.
Kloth L C, McCulloch J M. Promotion of wound healing with electrical stimulation. Adv Wound Care. 1996 September-October; 9(5):42-5.
Karba R, Dejan Semrov, Vodovnik L, Benko H, S'avrin R. D C electrical stimulation for chronic wound healing enhancement Part 1. Clinical study and determination of electrical field distribution in the numerical wound model. Bioelectrochemistry and Bioenergetics, Volume 43, Issue 2, August 1997, Pages 265-270.
Reger S I, Hyodo A, Negami S, Kambic H E, Sahgal V. Experimental wound healing with electrical stimulation. Artif Organs. 1999 May; 23(5):460-2.
McCaig C D, Rajnicek A M, Song B, Zhao M. Controlling cell behavior electrically: current views and future potential. Physiol Rev. 2005 July; 85(3):943-78.
Talebi G, Torkaman G, Firoozabadi M, Shariat S. Effect of anodal and cathodal microamperage direct current electrical stimulation on injury potential and wound size in guinea pigs. J Rehabil Res Dev. 2008; 45(1): 153-9.
Nuccitelli R, Nuccitelli P, Ramlatchan S, Sanger R, Smith P J S. Imaging the electric field associated with mouse and human skin wounds. Wound Repair and Regeneration. 2008; 16(3):432-441.
Balakatounis K C, Angoules A G. Low-intensity Electrical Stimulation in Wound Healing: Review of the Efficacy of Externally Applied Currents Resembling the Current of Injury. Eplasty. 2008; 8:e28.
Zhao M. Electrical fields in wound healing—An overriding signal that directs cell migration. Semin Cell Dev Biol. 2009 August; 20(6):674-82.
Liu X, Lee P Y, Ho C M, Lui V C, Chen Y, Che C M, Tam P K, Wong K K. Silver nanoparticles mediate differential responses in keratinocytes and fibroblasts during skin wound healing ChemMedChem. 2010 Mar. 1; 5(3):468-75.
Messerli M A, Graham D M. Extracellular Electrical Fields Direct Wound Healing and Regeneration Biol Bull. 2011, August; 221(1):79-92
Nuccitelli R, Nuccitelli P, Li C, Narsing S, Pariser D M, Lui K. The electric field near human skin wounds declines with age and provides a noninvasive indicator of wound healing. Wound Repair Regen. 2011 September-October; 19(5).
Thakral G, LaFontaine J, Najafi B, Talal T K, Kim P, Lavery L A. Electrical stimulation to accelerate wound healing. Diabetic Foot & Ankle. 2013; 4: 10.3402/dfa.v4i0.22081.
Reid B, Zhao M. The Electrical Response to Injury: Molecular Mechanisms and Wound Healing. Advances in Wound Care. 2014; 3(2): 184-201.
Ud-Din S, Bayat A Electrical Stimulation and Cutaneous Wound Healing: A Review of Clinical Evidence. Healthcare 2014, 2(4), 445-467.
Hunckler J, de Mel A. A current affair: electrotherapy in wound healing. J Multidiscip Healthc 2017; 10:179-194. Published 2017 Apr. 20. doi:10.2147/JMDH.S127207.
Reid B, Song B, Zhao M. Electric currents in Xenopus tadpole tail regeneration. Dev Biol. 2009 Nov. 1; 335(1): 198-207. doi: 10.1016/j.ydbio.2009.08.028.

Cell Modification Via Electrical Stimulation:
Becker R O, Murray D G. A method for producing cellular dedifferentiation by means of very small electrical currents Trans N Y Acad Sci 1967 March; 29(5):606-15
Harrington D B, Becker R O. Electrical stimulation of RNA and protein synthesis in the frog erythrocyte. Exp Cell Res. 1973 January; 76(1):95-8.
Becker R O, Flick A B, Becker A J. Iontopheretic system for stimulation of tissue healing and regeneration. U.S. Pat. No. 5,814,094 A. Sep. 29, 1998.
Becker R O. Effects of Electrically Generated Silver Ions on Human Cells and Wound Healing. Journal Electro- and Magnetobiology. Volume 19, 2000—Issue 1, Pages 1-19.
Becker R O. Induced dedifferentiation: a possible alternative to embryonic stem cell transplants. NeuroRehabilitation. 2002; 17(1):23-31.
Levin M. Bioelectric mechanisms in regeneration: unique aspects and future perspectives. Seminars in cell & developmental biology. 2009; 20(5):543-556.
Rouabhia M, Park H, Meng S, Derbali H, Zhang Z. Electrical stimulation promotes wound healing by enhancing dermal fibroblast activity and promoting myofibroblast transdifferentiation PLoS One. 2013 Aug. 19; 8(8):e71660.

Cyclic Amp Second Messenger:

Low Frequency Electric Field Stimulation:
Sontag W, Dertinger H. Response of cytosolic calcium, cyclic AMP, and cyclic GMP in dimethylsulfoxide-differentiated HL-60 cells to modulated low frequency electric currents. Bioelectromagnetics. 1998; 19(8):452-458.
Sontag W. Response of cyclic AMP by DMSO differentiated HL-60 cells exposed to electric interferential current after prestimulation. Bioelectromagnetics. 2004 April; 25(3): 176-84. doi: 10.1002/bem.10183.
Knedlitschek G, Noszvai-Nagy M, Meyer-Waarden H, Schimmelpfeng J, Weibezahn K F, Dertinger H. Cyclic AMP response in cells exposed to electric fields of different frequencies and intensities. Radiat Environ Biophys. 1994; 33(2):141-147.

Effects on Pain:
Woessner J. Blocking Out the Pain: Electric nerve block treatments for sciatic neuritis. Practical Pain Management. March/April 2002, Volume 2, Issue #2.
Liou J T, Liu F C, Hsin S T, Yang C Y, Lui P W. Inhibition of the cyclic adenosine monophosphate pathway attenuates neuropathic pain and reduces phosphorylation of cyclic adenosine monophosphate response element-binding in the spinal cord after partial sciatic nerve ligation in rats. Anesth Analg. 2007 December; 105(6): 1830-7.
Ross C L, Teli T, Flarrison B S. Effect of electromagnetic field on cyclic adenosine monophosphate (cAMP) in a human mu-opioid receptor cell model. Electromagn Biol Med. 2016; 35(3):206-213.
Wang Y Y, Wu S X, Zhou L, Huang J, Wang W, Liu X Y, Li Y Q Dose-related antiallodynic effects of cyclic AMP response element-binding protein-antisense oligonucleotide in the spared nerve injury model of neuropathic pain. Neuroscience. 2006; 139(3):1083-93. Epub 2006 Mar. 3.
Ma W, Quirion R. Increased phosphorylation of cyclic AMP response element-binding protein (CREB) in the superficial dorsal horn neurons following partial sciatic nerve ligation. Pain. 2001 September; 93(3):295-301.
Hurle M A, Goirigolzarri I, Valdizan E M. Involvement of the cyclic AMP system in the switch from tolerance into supersensitivity to the antinociceptive effect of the opioid sufentanil. Br J Pharmacol. 2000 May; 130(1):174-80.
Gu X, Bo J, Zhang W, Sun X, Zhang J, Yang Y, Ma Z. Intrathecal administration of cyclic AMP response element-binding protein-antisense oligonucleotide attenuates neuropathic pain after peripheral nerve injury and decreases the expression of N-methyl-D-aspartic receptors in mice. Oncol Rep. 2013 July; 30(1):391-8. Epub 2013 Apr. 30, 2005 June 29; 3:6.
Shao X-M, Sun J, Jiang Y-L, Liu B-Y, Shen Z, Fang F, Du J-Y, Wu Y-Y, Wang J-L, Fang J-Q Inhibition of the cAMP/PKA/CREB Pathway Contributes to the Analgesic Effects of Electroacupuncture in the Anterior Cingulate Cortex in a Rat Pain Memory Model. Neural Plast. 2016; 2016: 5320641.
Brust T F, Alongkronrusmee D, Soto-Velasquez M, Baldwin T A, Ye Z, Dai M, Dessauer C W, Van Rijn R M, Watts V J. Identification of a selective small-molecule inhibitor of type 1 adenylyl cyclase activity with analgesic properties. Sci Signal. 2017 Feb. 21; 10(467).
Fitzgerald E M, Okuse K, Wood J N, Dolphin A C, Moss S J. cAMP-dependent phosphorylation of the tetrodotoxin-resistant voltage-dependent sodium channel SNS. J Physiol. 1999 Apr. 15; 516 (Pt 2):433-46.
Liu L, Yang T, Bruno M J, Andersen O S, Simon S A. Voltage-gated ion channels in nociceptors: modulation by cGMP. J Neurophysiol. 2004 October; 92(4):2323-32. Epub 2004 Jun. 2.
Sluka K A. Activation of the cAMP transduction cascade contributes to the mechanical hyperalgesia and allodynia induced by intradermal injection of capsaicin. Br J Pharmacol. 1997 November; 122(6): 1165-73.
Lee L Y, Kwong K, Lin Y S, Gu Q. Hypersensitivity of bronchopulmonary C-fibers induced by airway mucosal inflammation: cellular mechanisms. Pulm Pharmacol Ther. 2002; 15(3): 199-204.
Gu Q, Ruan T, Hong J-L, Burki N, Lee L-Y. Hypersensitivity of pulmonary C fibers induced by adenosine in anesthetized rats. J Appl Physiol, 1 Sep. 2003.
Middlekauff H R, Doering A, Weiss J N. Circulation. Adenosine enhances neuroexcitability by inhibiting a slow postspike after hyperpolarization in rabbit vagal afferent neurons. 2001 Mar. 6; 103(9): 1325-9.

Nerve Tissue Regeneration:
Li M, Wang X, Meintzer M K, Laessig T, Birnbaum M J, Heidenreich K A. Cyclic AMP promotes neuronal survival by phosphorylation of glycogen synthase kinase 3beta. Mol Cell Biol. 2000 December; 20(24):9356-63.
Cai D, Qiu J, Cao Z, McAtee M, Bregman B S, Filbin M T. Neuronal cyclic AMP controls the developmental loss in ability of axons to regenerate. J Neurosci. 2001 Jul. 1; 21 (13):4731-9.
Lau B Y, Fogerson S M, Walsh R B, Morgan J R. Cyclic AMP promotes axon regeneration, lesion repair and neuronal survival in lampreys after spinal cord injury. Exp Neurol. 2013 December; 250:31-42. Epub 2013 Sep. 13.
Knott E P, Assi M, Pearse D D. Cyclic AMP Signaling: A Molecular Determinant of Peripheral Nerve Regeneration. BioMed Research International. Volume 2014 (2014).
Dugan L L, Kim J S, Zhang Y, Bart R D, Sun Y, Holtzman D M, Gutmann D H. Differential effects of cAMP in neurons and astrocytes. Role of B-raf. J Biol Chem. 1999 Sep. 3; 274(36):25842-8.
Ghosh-Roy A, Wu Z, Goncharov A, Jin Y, Chisholm A D. Calcium and cyclic AMP promote axonal regeneration in Caenorhabditis elegans and require DLK-1 kinase. J Neurosci. 2010 Mar. 3; 30(9):3175-83. doi: 10.1523/JNEUROSCI.5464-09.2010.
Hannila S S, Filbin M T. The role of cyclic AMP signaling in promoting axonal regeneration after spinal cord injury. Exp Neurol. 2008 February; 209(2):321-32. Epub 2007 Aug. 27.
Qiu J, Cai D, Dai H, McAtee M, Hoffman P N, Bregman B S, Filbin M T. Spinal axon regeneration induced by elevation of cyclic AMP. Neuron. 2002 Jun. 13; 34(6):895-903.

Effects on Inflammation:
Erdogan S, Aslantas O, Celik S, Atik E. The effects of increased cAMP content on inflammation, oxidative stress and PDE4 transcripts during Brucella melitensis infection. Res Vet Sci 2008 February; 84(1): 18-25 Epub 2007 Mar. 29
Yuan X, Arkonac D E, Chao P-H G, Gordana V N. Electrical stimulation enhances cell migration and integrative repair in the meniscus. Nature, Scientific Reports 4, Article number: 3674 (2014).
Hoyle G W. Mitigation of chlorine lung injury by increasing cyclic AMP levels. Proc Am Thorac Soc. 2010 July; 7(4):284-9.
Ji H, Shen X D, Zhang Y, Gao F, Huang C Y, Chang W W, Lee C, Ke B, Busuttil R W, Kupiec-Weglinski J W. Activation of cyclic adenosine monophosphate-dependent protein kinase a signaling prevents liver ischemia/reperfusion injury in mice. Liver Transpl. 2012 June; 18(6):659-70.
Sakaguchi T, Asai T, Belov D. Okada M, Pinsky D J, Schmidt A M, Naka Y. Influence of ischemic injury on vein graft remodeling: Role of cyclic adenosine monophosphate second messenger pathway in enhanced vein graft preservation. J Thorac Cardiovasc Surg. Volume 129, Issue 1, January 2005, Pages 129-137.

Effects on Circulation:
Bubb K J, Trinder S L, Baliga R S, Patel J, Clapp L H, MacAllister R J, Hobbs A J. Inhibition of phosphodiesterase 2 augments cGMP and cAMP signaling to ameliorate pulmonary hypertension. Circulation. 2014 Aug. 5; 130(6):496-507.
Tseng S Y, Chao T H, Li Y H, Liu P Y, Lee C H, Cho C L, Wu H L, Chen J H. Cilostazol improves high glucose-induced impaired angiogenesis in human endothelial progenitor cells and vascular endothelial cells as well as enhances vasculoangiogenesis in hyperglycemic mice mediated by the adenosine monophosphate-activated protein kinase pathway. J Vase Surg. 2016 April; 63(4): 1051-62. e3.
Stott J, Greenwood I. Complex role of Kv7 channels in cGMP and cAMP-mediated relaxations. Channels. 2015; 9(3): 117-118.
Garcia-Morales V, Cuihas A, Elies J, Campos-Toimil M. PKA and Epac activation mediates cAMP-induced vasorelaxation by increasing endothelial NO production. Vascul Pharmacol. 2014 March; 60(3):95-101.
Morales-Cano D, Moreno L, Barreira B, Briones A M, Pandolfi R, Moral-Sanz J, Callejo M, Mondejar-Parreno G, Cortijo J, Salaices M, Duarte J, Perez-Vizcaino F, Cogolludo A. Activation of RRARb/d prevents hyperglycaemia-induced impairment of Kv7 channels and cAMP-mediated relaxation in rat coronary arteries. Clin Sci (Lond). 2016 Oct. 1; 130(20): 1823-36. Clin Sci (Lond). 2016 Oct. 1; 130(20): 1823-36.
Wong K H, Truslow J G, Tien J. The role of cyclic AMP in normalizing the function of engineered human blood microvessels in microfluidic collagen gels. Biomaterials. 2010 June; 31(17):4706-14.

Injured Muscle Tissue Regeneration:
Stewart R, Flechner L, Montminy M, Berdeaux R. CREB is activated by muscle injury and promotes muscle regeneration. PLoS One. 2011; 6(9):e24714. Epub 2011 Sep. 13.
Kerrick W G, Hoar P E. Inhibition of smooth muscle tension by cyclic AMP-dependent protein kinase. Nature. 1981 Jul. 16; 292(5820):253-5.
Berdeaux R, Stewart R. cAMP signaling in skeletal muscle adaptation: hypertrophy, metabolism, and regeneration. Am J Physiol Endocrinol Metab. 2012 Jul. 1; 303(1):E1-17. Epub 2012 Feb. 21.
Young R B, Bridge K Y, Strietzel C J. Effect of electrical stimulation on beta-adrenergic receptor population and cyclic amp production in chicken and rat skeletal muscle cell cultures. In Vitro Cell Dev Biol Anim. 2000 March; 36(3): 167-73.

Wound Healing:
Kim M O, Ryu J M, Suh H N, Park S H, Oh Y M, Lee S H, Han H J. cAMP Promotes Cell Migration Through Cell Junctional Complex Dynamics and Actin Cytoskeleton
Remodeling: Implications in Skin Wound Healing. Stem Cells Dev. 2015 Nov. 1; 24(21):2513-24.
Kim W K, Song S Y, Oh W K, Kaewsuwan S, Tran T L, Kim W S, Sung J H. Wound-healing effect of ginsenoside Rd from leaves of Panax ginseng via cyclic AMP-dependent protein kinase pathway. Eur J Pharmacol. 2013 Feb. 28; 702(1-3):285-93.
Pullar C E, Isseroff R R. Cyclic AMP mediates keratinocyte directional migration in an electric field. J Cell Sci. 2005 May 1; 118(Pt 9):2023-34.
Zhu K, Sun Y, Miu A, Yen M, Liu B, Zeng Q, Mogilner A, Zhao M. cAMP and cGMP Play an Essential Role in Galvanotaxis of Cell Fragments. J Cell Physiol. 2016 June; 231(6):1291-300.
Shibata H, Shioya N, Kuroyanagi Y. Development of new wound dressing composed of spongy collagen sheet containing dibutyryl cyclic AMP. J Biomater Sci Polym Ed. 1997; 8(8):601-21.
Aghajanian J, Hand A, Genutis S, Tatch W, Mednieks M Expression of Cyclic AMP-Receptor (cARP) Proteins by Fibroblasts in Culture: Effects of Mechanical Disruption. Microsc Microanal 9(Suppl 2), 2003, pp 1386-1387.
Rundfeldt C, Steckel H, Sorensen T, Wlazcorresponding P. The stable cyclic adenosine monophosphate analogue, dibutyryl cyclo-adenosine monophosphate (bucladesine), is active in a model of acute skin inflammation. Arch Dermatol Res. 2012 May; 304(4): 313-317.
Balakrishnan B, Mohanty M, Fernandez A C, Mohanan P V, Jayakrishnan A. Evaluation of the effect of incorporation of dibutyryl cyclic adenosine monophosphate in an in situ-forming hydrogel wound dressing based on oxidized alginate and gelatin. Biomaterials. 2006 March; 27(8):1355-61.
Lu D, Aroonsakool N, Yokoyama U, Patel H H, Insel P A. Increase in cellular cyclic AMP concentrations reverses the profibrogenic phenotype of cardiac myofibroblasts: a novel therapeutic approach for cardiac fibrosis Mol Pharmacol. 2013 December; 84(6):787-93.
Wong K H, Truslow J G, Tien J. The role of cyclic AMP in normalizing the function of engineered human blood microvessels in microfluidic collagen gels. Biomaterials. 2010 June; 31(17):4706-14.
Busca R, Ballotti R. Cyclic AMP a key messenger in the regulation of skin pigmentation. Pigment Cell Res 2000; 13:60-9. Pigment Cell Res. 2000 April; 13(2):60-9.
Jumblatt M M, Neufeld A H. Characterization of cyclic AMP-mediated wound closure of the rabbit corneal epithelium. Curr Eye Res. 1981; 1(4): 189-95.

Effects in Infection:
Ramdani G, Naissant B, Thompson E, Breil F, Lorthiois A, Dupuy F, Cummings R, Duffier Y, Corbett Y, Mercereau-Puijalon O, Vernick K, Taramelli D, Baker D A, Langsley G, Lavazec C. cAMP-Signalling Regulates Gametocyte-Infected
Erythrocyte Deformability Required for Malaria Parasite Transmission. PLoS Pathog. 2015 May 7; 11 (5):e1004815.
Molina-Quirez R C, S-V Cecilia, Brewster J, C-N Eduardo, Levy S B, Camilli A. Cyclic AMP Regulates Bacterial Persistence through Repression of the Oxidative Stress Response and SOS-Dependent DNA Repair in Uropathogenic Escherichia coli. mBio January 2018, 9 (1) e02144-17; DOI: 10.1128/mBio.02144]

Cellular Apoptosis Effects:
Fajardo A M, Piazza G A, Tinsley H N The Role of Cyclic Nucleotide Signaling Pathways in Cancer: Targets for Prevention and Treatment. Cancers (Basel). 2014 Feb. 26; 6(1):436-58.
Gottesman M M, Fleischmann R D. The role of cAMP in regulating tumour cell growth. Cancer Surv. 1986; 5(2):291-308.
Marko D, Romanakis K, Zankl H, Furstenberger G, Steinbauer B, Eisenbrand G. Induction of apoptosis by an inhibitor of cAMP-specific PDE in malignant murine carcinoma cells overexpressing PDE activity in comparison to their nonmalignant counterparts. Cell Biochem Biophys. 1998; 28(2-3):75-101.
Loffler I, Grun M, Bohmer F D, Rubio I. Role of cAMP in the promotion of colorectal cancer cell growth by prostaglandin E2. BMC Cancer. 2008 Dec. 19; 8:380.
Sheffield L G, Welsch C W. Cholera-toxin-enhanced growth of human breast cancer cell lines in vitro and in vivo: interaction with estrogen. Int J Cancer. 1985 Oct. 15; 36(4):479-83.
Lerner A, Kim D H, Lee R. The cAMP signaling pathway as a therapeutic target in lymphoid malignancies. Leuk Lymphoma. 2000 March; 37(1-2):39-51.
Murray F, Insel P A. Targeting cAMP in chronic lymphocytic leukemia: a pathway-dependent approach for the treatment of leukemia and lymphoma. Expert Opin Ther Targets 2013 August; 17(8):937-49. Epub 2013 May 7
Insel P A, Wilderman A, Zhang L, Keshwani M M, Zambon A C Cyclic AMP/PKA-promoted apoptosis: insights from studies of S49 lymphoma cells. Horm Metab Res. 2014 November; 46(12):854-62 Epub 2014 Jul. 16.

Cyclic Gmp Second Messenger:

Wound Healing:
Zhan R, Yang S, He W, Wang F, Tan J, Zhou J, Yang S, Yao Z, Wu J, Luo G. Nitric oxide enhances keratinocyte cell migration by regulating Rho GTPase via cGMP-PKG signalling. PLoS One. 2015 Mar. 23; 10(3):e0121551. doi: 10.1371/journal. pone.0121551. eCollection 2015.
Zhan R, He W, Wang F, et al. Nitric oxide promotes epidermal stem cell migration via cGMP-Rho GTPase signalling. Sci Rep 6, 30687 (2016). https://doi.Org/10.1038/srep30687.
Korotkina R N, Nosova I M, Zaidenberg M A, Karelin A A. Effect of cyclic guanosine monophosphate on certain indices of muscle tissue carbohydrate metabolism during the wound process. Biull Eksp Biol Med. 1980 September; 90(9):361-3.
Abaffy P, Tomankova S, Naraine R, Kubista M, Sindelka R. The role of nitric oxide during embryonic wound healing. BMC Genomics. 2019 Nov. 6; 20(1):815. doi: 10.1186/si 2864-019-6147-6.
Spitler R, Schwappacher R, Wu T, Kong X, Yokomori K, Pilz R B, Boss G R, Berns M W. Nitrosyl-cobinamide (NO-Cbi), a new nitric oxide donor, improves wound healing through cGMP/cGMP-dependent protein kinase. Cell Signal. 2013 December; 25(12):2374-82. doi: 10.1016/j cellsig.2013.07.029. Epub 2013 Aug. 3.
Chen Y J, Wu S C, Wang H C, Wu T H, Yuan S F, Lu T T, Liaw W F, Wang Y M. Activation of Angiogenesis and Wound Healing in Diabetic Mice Using NO-Delivery Dinitrosyl Iron Complexes. Mol Pharm. 2019 Oct. 7; 16(10):4241-4251. doi: 10.1021/acs.molpharmaceut.9b00586. Epub 2019 Sep. 5.

1. A multi-type combination electrical stimulation method of medical treatment of any one of: surface and internal infected tissues, surface wounds and ulcers, trauma injuries, abnormal plasia and fibrotic changes, and pain conditions, the method comprising the steps of:
providing a vector bioparameter monitoring, measuring and electrical stimulation device, connected to measurement and stimulation parameter integrated material construction electrode pads, capable of multi-type combination electrical stimulation;
placing the electrode pads across or anatomically cross-sectioning or bioelectrically topographically matching a target location being a target physical lesion, tissue or anatomical structure;
measuring a bioelectric or biochemical vector bioparameter of the target location using the device and electrode pads at repeating intervals to generate measurement data, analytically calculating vector bioparameter dynamics data from the measurement data;
concurrently repeatedly measuring and analytically calculating compensation data that represent the electrochemical, electromechanical, environmental and body temperature, humidity, and skin condition changes affecting the measurement and stimulation electrode pad interfaces with the body using the device and electrode pads, applying the compensation data to the vector bioparameter dynamics data to generate compensated vector bioparameter dynamics data that represent the actual physiological change of the vector bioparameter separated from the compensation data; and
determining a stimulation time period that relates to the vector bioparameter at the target location, the selection, timing and sequencing of the multi-type combination and parameter electrical stimulation of the target location during the next stimulation time period then being determined by the compensated vector bioparameter dynamics data of the previous stimulation time period and statistically and analytically across multiple previous stimulation time periods, resulting from the effects of the previous multi-type electrical stimulation combinations and parameters applied to the target location, and compared to a biologically and theoretical modeled improving dynamic of the vector bioparameter of the specific lesion or pathology at the target location.

2. The method of claim 1, wherein the multi-type electrical stimulation combinations generated by the device are comprised of low intensity direct constant current, constant millivolt voltage peak amplitude modulated waveform, and constant millivolt static electric field with reversible polarity, all with variable ranges and parameters.

3. The method of claim 2, wherein the waveform type of electrical stimulation comprises generating sequenced and timed carrier base frequency waveforms with amplitude modulation of the carrier base waveforms by secondary frequencies.

4. The method of claim 1, wherein the durations of the measurement periods and intervals of the vector bioparameter measurement data and of the compensation data, and the duration of the stimulation time period, are separately adjusted to the instrument detectable limits and measurement resolution, and actual rates of changes, to enable the separated measurements and acquisition of the data sets.

5. The method of claim 1, wherein the repeated selection of effective multi-type electrical stimulation combinations and parameters continuously improves based on previous compensated bioparameter dynamics data behavior under stimulation, with changes in the target location over time and its physiological and pathological course and events.

6. The method of claim 5, wherein assessment of the compensated vector bioparameter dynamics data and the compensation data changes are performed with continuously updated regression analysis or by other statistical and analytical means.

7. The method of claim 2, wherein all possible multi-type electrical stimulation and parameter combinations are derived from a switch set table comprising top level binary switch states of stimulation type on or off, and non binary second level switch states consisting of the full ranges of parameters of each of the different electrical stimulation types.

8. The method of claim 3, wherein during waveform type electrical stimulation the device generated carrier base waveform frequencies are in the range of 1-20,000 Hz and the amplitude modulating signal frequencies are in the range of 1-200 Hz.

9. The method of claim 3, wherein the amplitude modulated waveforms, sequencing and timings generated by the stimulation device are utilized as specific bioinformational codes that select and target the regulation of individual intracellular second messengers.

10. The method of claim 8, wherein the carrier base waveform frequency is calculated based on the previously measured overall frequency dependent complex impedance profile of the internal body tissues between the electrode pads, and wherein the voltage peak amplitude of the carrier base waveform is repeatedly and automatically analytically calculated and varied by the device based on the complex impedance profile to maintain a constant resultant alternating current through the body and the target location.

11. The method of claim 9, wherein the second messengers include are comprised of: cyclic AMP: adenosine 3′,5′-monophosphate, and cyclic GMP: guanosine 3′,5′-cyclic monophosphate.

12. The method of claim 9, wherein the stimulation device generates a repeating, timed stimulation sequence and cycle that includes a signal frequency amplitude modulated carrier base frequency waveform that signals the up-regulation of production of the specific second messenger, followed by a first rest period of variable duration.

13. The method of claim 12, wherein the step is followed by the carrier base frequency waveform differently signal frequency amplitude modulated that signals the activation and utilization of the specific second messenger, then followed by a second rest period also of variable duration.

14. The method of claim 9, wherein the carrier base frequency waveform is amplitude modulated by a signal frequency of 10 Hz for up-regulating production, followed by the same carrier base frequency waveform amplitude modulated by a signal frequency of 20 Hz for increasing utilization of cyclic AMP; and
wherein the carrier base frequency waveform is signal frequency modulated by 25 Hz for up-regulating production, followed by the same carrier base frequency waveform signal frequency amplitude modulated by 20 Hz for increasing utilization of cyclic GMP.

15. The method of claim 12, wherein the rest periods are 1-3 minutes duration that empirically critically enable and optimize the overall intracellular second messenger biostimulation.
